(19) United States
(12) Patent Application Publication
(10) Pub. No.: US 2026/0340816 A1
(43) Pub. Date: Oct. 29, 2026

(54) ULTRASOUND VIDEO FEATURE DETECTION USING LEARNING FROM UNLABELED DATA

(71) Applicant: KONINKLIJKE PHILIPS N.V., EINDHOVEN (NL)

(72) Inventors: Li Chen, Cambridge, MA (US); Alvin Chen, Cambridge, MA (US)

(21) Appl. No.: 19/106,310

(22) PCT Filed: Aug. 22, 2023
(86) PCT No.: PCT/EP2023/072994

§ 371 Date: Feb. 25, 2025

Related U.S. Application Data
(60) Provisional application No. 63/402,152, filed on Aug. 30, 2022.

(51) Int. Cl. G06T 7/00; A61B 8/00; A61B 8/08

(52) U.S. Cl. CPC G06T 7/0012; A61B 8/08; A61B 8/463; A61B 8/54; G06T 2207/10016; G06T 2207/10132; G06T 2207/20081; G06T 2207/20084; G06T 2207/30004

(57) ABSTRACT

A processor trains a neural network using self-supervised learning with a first augmented ultrasound video and a second augmented ultrasound video. The processor performs spatial and/or temporal augmentation(s) to frame(s) of an unlabeled ultrasound video to generate the first and second augmented ultrasound videos. The training is based on comparing outputs associated with the first and second augmented ultrasound videos to one another. The neural network is implemented during inference to identify pathology associated with the anatomy. A processor generates a heat map identifying a location of a pathology in frame(s) of an ultrasound video using the neural network and provides, to a display, a screen display with the heat map.

### FIELD

The subject matter described herein relates to devices, systems, and methods for locating and visualizing features (e.g., anatomical features, such as pathology) in frames of an ultrasound video. The features are detected using a machine learning (ML) algorithm, such as a neural network, that is trained with self-supervised learning (SSL) using unlabeled ultrasound videos have that have spatially and/or temporally augmented.

### BACKGROUND

Ultrasound imaging is often used for diagnostic purposes in an office or hospital setting. For example, lung ultrasound (LUS) is an imaging technique deployed at the point-of-care to aid in evaluation of pulmonary and infectious diseases, including COVID-19 pneumonia. Important clinical features—such as B-lines, merged B-lines, pleural line changes, consolidations, and pleural effusions-can be visualized under LUS, but accurately identifying these clinical features can be a challenging skill. Automated identification and visualization of sonographic features by machine learning models is thus beneficial.

However, data and annotation challenges for artificial intelligence (AI) and machine learning (ML) applications are formidable. For example, AI algorithms developed on medical images require extensive training data with expert human annotations of high clinical quality. For ultrasound in particular, frame-by-frame annotation of a single cineloop video can take hours for a trained clinician to complete, which makes annotation-at-scale both challenging and expensive. Thus, despite the recent success of lung ultrasound algorithms, such as auto B-line detection, further development of these AI tools requires extensive manual annotation efforts.

The information included in this Background section of the specification, including any references cited herein and any description or discussion thereof, is included for technical reference purposes only and is not to be regarded as subject matter by which the scope of the disclosure is to be bound.

### SUMMARY

Disclosed is an ultrasound video feature detection system with a machine learning algorithm (e.g., a neural network) trained with self-supervised learning (SSL) using unlabeled data having spatial and/or temporal augmentations. This SSL ultrasound video feature detection system disclosed herein has particular, but not exclusive, utility for finding pixels that are likely to have features of interest (e.g., pathology) within the frames of an ultrasound video, such as a lung ultrasound video. The SSL ultrasound video feature detection system includes a training mode, in which the machine learning algorithm is trained using unlabeled ultrasound video data. Unlabeled ultrasound video does not have ground truth annotation by an expert user. Instead, the SSL is performed with spatial and/or temporal augmentations on the ultrasound video. The SSL ultrasound video feature detection system also includes an inference mode, in which the machine learning algorithm generates a heat map identifying pixels of interest (e.g., suspected pathology) within frame(s) of ultrasound video. This heat map may for example be overlaid on the video.

In an exemplary aspect, a system is provided. The system includes a display; and a processor configured for communication with the display, wherein the processor is configured to: receive an ultrasound video of anatomy obtained by an ultrasound probe, wherein ultrasound video comprises a plurality of frames; provide the ultrasound video to a neural network during inference, wherein the neural network is trained with self-supervised learning using unlabeled ultrasound videos comprising at least one of a plurality of spatial augmentations or a plurality of temporal augmentations; generate, using the neural network, a heat map identifying a location of a pathology in one or more frames of the plurality of frames; and provide, to the display, a screen display comprising the heat map.

In some aspects, the neural network is trained using only unlabeled ultrasound videos. In some aspects, the screen display further comprises the ultrasound video. In some aspects, the heat map is overlaid on the one or more frames of the plurality of frames of the ultrasound video. In some aspects, the heat map comprises a single heat map, and the single heat map is overlaid on each of the plurality of frames of the ultrasound video. In some aspects, the processor is configured to generate a plurality of heat maps, and the screen display comprises the plurality of heat maps respectively overlaid on plurality of frames of the ultrasound video. In some aspects, a direct output of the neural network comprises a visual representation associated the one or more frames of the ultrasound video, and dimensions of the visual representation are different than dimensions of the plurality of frames of the ultrasound video. In some aspects, to generate the heat map, the processor is configured to convert the dimensions of the visual representation to the dimensions of the ultrasound video. In some aspects, the processor is configured to: provide the visual representation to a further neural network configured for at least one of classification, regression, object detection, or segmentation; and generate an output using the further neural network. The output is associated with at least one of the classification, the regression, the object detection, or the segmentation. The output is different than the visual representation and the heat map. The screen display comprises a visualization based on the output. In some aspects, the system further includes the ultrasound probe, and the processor is configured to control the ultrasound probe to obtain the ultrasound video.

In an exemplary aspect, a system is provided. The system includes a memory comprising an ultrasound video of anatomy obtained by an ultrasound probe, wherein the ultrasound video is unlabeled and comprises a plurality of frames; and a processor configured for communication with the memory, wherein the processor is configured to: retrieve the ultrasound video from the memory; perform a first augmentation to the ultrasound video to generate a first augmented ultrasound video; perform a second augmentation to the ultrasound video to generate a second augmented ultrasound video, wherein the first augmentation and the second augmentation comprise at least one of a spatial augmentation to the plurality of frames or a temporal augmentation to the plurality of frames; train a neural network with a first plurality of weights, using the first augmented ultrasound video and the second augmented ultrasound video, wherein the training of the neural network comprises self-supervised learning; and provide, after the training, the neural network with a second plurality of weights, wherein the neural network with the second plurality of weights is configured to be implemented during inference to identify pathology associated with the anatomy, wherein, to train the neural network, the processor is configured to: generate a first output associated with the neural network based on the first augmented ultrasound video; generate a second output associated with the neural network based on the second augmented ultrasound video; and determine the second plurality of weights based on a comparison between the first output and the second output.

In some aspects, to train the neural network, the processor is configured to implement a first processing path associated with first augmented ultrasound video and a second processing path associated with the second augmented ultrasound video. In some aspects, the memory further comprises a plurality of ultrasound videos, and the processor is configured to: obtain the plurality of ultrasound videos from the memory; and train the neural network based on the plurality of ultrasound videos, and a majority of the plurality of ultrasound videos are unlabeled. In some aspects, all of the plurality of ultrasound videos are unlabeled. In some aspects, the spatial augmentation comprises a change to how image content is depicted in the plurality of frames, and the temporal augmentation comprises a change to an order in which the plurality of frames are arranged. In some aspects, the first augmentation is different than the second augmentation, and the ultrasound video, the first augmented ultrasound video, and the second augmented ultrasound video are different from one another. In some aspects, the first output associated with the neural network comprises a first one-dimensional (1D) set of projected features, the second output associated with the neural network comprises a second 1D set of projected features, and the processor is configured to determine the second plurality of weights to maximize agreement between the first 1D set of projected features and the second 1D set of projected features. In some aspects, to train the neural network, the processor is configured to: generate a first initial output of the neural network based on the first augmented ultrasound video, before the first output is generated; and generate a second initial output of the neural network based on the second augmented ultrasound video, before the second output is generated, the first initial output comprises a first plurality of two-dimensional (2D) visual representations, and the second initial output comprises a second plurality of 2D visual representations. In some aspects, to train the neural network, the processor is configured to: compress the first plurality of 2D visual representations using a further neural network to generate the first 1D set of projected features, and compress the second plurality of 2D visual representations using the further neural network to generate the second 1D set of projected features. In some aspects, the neural network comprises a convolutional neural network, and the further neural network comprises a multilayer perceptron.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the SSL ultrasound video feature detection system, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a schematic, diagrammatic representation of an ultrasound imaging system, in accordance with at least one aspect of the present disclosure.

FIG. 2 is a schematic diagram of a processor circuit, according to aspects of the present disclosure.

FIG. 3 is a schematic, diagrammatic representation of a radiology video clip, in accordance with at least one aspect of the present disclosure.

FIG. 4 is a schematic, diagrammatic representation of a labeled ultrasound data set, in accordance with at least one aspect of the present disclosure.

FIG. 5 is a schematic, diagrammatic representation of an unlabeled ultrasound data set, in accordance with at least one aspect of the present disclosure.

FIG. 6 is a schematic, diagrammatic representation of the extraction of sub-clips from an ultrasound video clip, in accordance with at least one aspect of the present disclosure.

FIG. 7 is a schematic, diagrammatic illustration of a 3D spatial-temporal augmentation method for ultrasound video data, in accordance with at least one aspect of the present disclosure.

FIG. 8 is a schematic, diagrammatic overview, in block diagram form, of a self-supervised learning mode for the SSL ultrasound video feature detection system, in accordance with at least one aspect of the present disclosure.

FIG. 9 is a is a schematic, diagrammatic overview, in block diagram form, of a self-supervised learning mode for the SSL ultrasound video feature detection system, in accordance with at least one aspect of the present disclosure.

FIG. 10 is a flow diagram of an example SSL ultrasound video feature detection system training method, according to at least one aspect of the present disclosure.

FIG. 11 is a schematic, diagrammatic overview, in block diagram form, of an inference mode for the SSL ultrasound video feature detection system, in accordance with at least one aspect of the present disclosure.

FIG. 12 is a schematic, diagrammatic overview, in block diagram form, of an inference mode for the SSL ultrasound video feature detection system, in accordance with at least one aspect of the present disclosure.

FIG. 13 is a schematic, diagrammatic overview, in block diagram form, of an inference mode for the SSL ultrasound video feature detection system, in accordance with at least one aspect of the present disclosure.

FIG. 14 is a schematic, diagrammatic view of a formatted heat map being overlaid on an incoming video frame to yield a highlighted output frame, in accordance with at least one aspect of the present disclosure.

FIG. 15 is a flow diagram of an example inference-mode SSL video annotation method, according to at least one aspect of the present disclosure.

FIG. 16 is an exemplary representation of the highlighting of features in a video frame by a supervised learning algorithm, in accordance with at least one aspect of the present disclosure.

FIG. 17 is an exemplary representation of highlighting of features in a video frame by a self-supervised learning (SSL) algorithm, in accordance with at least one aspect of the present disclosure.

FIG. 18 is a graph showing test accuracy for partially trained machine learning models, in accordance with at least one aspect of the present disclosure.

FIG. 19 is a graph showing the average of test sensitivity and test specificity for partially trained machine learning models, in accordance with at least one aspect of the present disclosure.

FIG. 20 is a graph showing test area under the curve (AUC) for partially trained machine learning models, in accordance with at least one aspect of the present disclosure.

FIG. 21 is a schematic, diagrammatic overview, in block diagram form, of an inference mode for the SSL ultrasound video feature detection system, in accordance with at least one aspect of the present disclosure.

FIG. 22 is a schematic, diagrammatic overview, in block diagram form, of an inference mode for the SSL ultrasound video feature detection system, in accordance with at least one aspect of the present disclosure.

FIG. 23 is a schematic, diagrammatic overview, in block diagram form, of an inference mode for the SSL ultrasound video feature detection system, in accordance with at least one aspect of the present disclosure.

FIG. 24 is an example screen display that includes both the highlighted image frame and a classification output, in accordance with at least one aspect of the present disclosure.

FIG. 25 is an example screen display that includes both the highlighted image frame and a regression output (e.g., a numerical score indicating the probability or severity of a detected pathology), in accordance with at least one aspect of the present disclosure.

FIG. 26 is an example screen display that includes both the highlighted image frame and an object detection output (e.g. a bounding box indicating a region of interest), in accordance with at least one aspect of the present disclosure.

FIG. 27 is a schematic, diagrammatic overview, in block diagram form, of at least a portion of a training mode for the SSL ultrasound video feature detection system, in accordance with at least one aspect of the present disclosure.

### DETAILED DESCRIPTION

In accordance with at least one aspect of the present disclosure, a self-supervised learning (SSL) video annotation system is provided which can identify features of clinical interest in a radiology video such as a lung ultrasound, and which can be trained using little or no human-annotated data. Other types of radiology videos may include X-ray videos, camera videos, computer-aided tomography (CT) scans, magnetic resonance imaging (MRI) scans, etc.

Accurately identifying clinical features in ultrasound video can be a challenging skill. Automated identification and visualization of sonographic features by machine learning models is thus beneficial. However, supervised learning for an AI or ML algorithm requires large amounts of frame-by-frame, human-annotated data, which is time-consuming and expensive to develop, especially for video data. It may therefore be substantially easier to obtain large amounts of unlabeled medical image data than it is to create a properly labeled dataset. Thus, unlabeled training data often outnumbers labeled training examples by a factor of 100:1 or more.

Self-supervised learning (SSL) methods are a promising alternative to standard (supervised) AI training because these methods require no or very limited labeled data.

Instead, SSL methods utilize unlabeled data to learn meaningful visual representations, relying on heavy data augmentation during training in the absence of ground-truth. Variants of SSL have been described in the art for processing of 2D natural images and medical images. SSL has also been combined with traditional supervised training for 2D images, leveraging labeled and unlabeled training datasets together to improve prediction performance.

Applying SSL to ultrasound videos is challenging. Currently, successful applications using SSL have been reported on 2D natural images and use 2D data augmentation techniques suited for natural imagery. Different from natural images, ultrasound data includes video sequences (3D data format, including an X-axis, Y-axis, and time axis) with many factors affecting image quality, including scan settings, scan angles, probe contact, probe motion, body habitus, and numerous other factors.

For example, applying SSL on lung ultrasound videos for medical applications (e.g., detection of consolidations associated with COVID-19 infection) is not a trivial task. In the present disclosure, SSL models to train on ultrasound data are developed using a specialized data augmentation process that simulates the full variability seen in ultrasound imagery. Furthermore, the data augmentation and model architecture are compatible with videos as opposed to 2D images. Such methods are novel in the art for ultrasound video sequences.

To apply SSL on the challenging lung ultrasound application, the SSL ultrasound video feature detection system employs a 3D augmentation method based on joint spatial and temporal image transformations that simulate the varied image appearances and scanning scenarios in the real-world setting. The SSL ultrasound video feature detection system combines the 3D augmentation method with an SSL training process to generate meaningful visual representations (such as heat maps indicating clinical significance of different regions) without the need for labeled data during training. The visual representations are then up-sampled to the resolution of the original image and displayed to the user. An AI model trained in this manner can highlight regions of potential clinical relevance within each frame of an ultrasound video. The AI model can be developed with no or limited annotation effort.

Automated identification and highlighting of relevant features in ultrasound videos, e.g. based on AI, can aid in clinical decision-making. The present disclosure therefore provides a system that locates and visualizes relevant features in ultrasound videos by learning from unlabeled data. The SSL ultrasound video feature detection system utilizes a self-supervised learning (SSL) method in combination with spatial-temporal 3D video augmentations to generate meaningful visual maps from radiology videos such as ultrasound video clips or cineloops. In the software end-product, highlighted regions detected with high probability by the SSL-trained model are displayed as heat maps to the user. These heat maps are learned directly from the video data and do not require manual annotations during training. The system may for example be trained with lung ultrasound video cineloops, and can generate heat maps from live or stored video that are strongly associated with regions of pathology (e.g., consolidations, pleural effusion, etc.) in the ultrasound video, indicating their usefulness for computer-aided detection.

Thus, the SSL ultrasound video feature detection system locates and visualizes relevant features in ultrasound videos by learning from unlabeled data, which is enabled through the use of 3D video augmentations within a self-supervised learning (SSL) training process that requires no or very limited ground-truth annotations. The input is an ultrasound cineloop, and the output is a heat map for each frame of the cineloop, highlighting regions detected by the SSL. The heat maps may be overlayed on the B-mode ultrasound for display.

A proper data augmentation procedure is a key module for SSL training.

Different from natural 2D images, lung ultrasound is fundamentally a 3D temporal data type (e.g., X-Y-time). The SSL ultrasound video feature detection system introduces a 3D augmentation method that applies joint spatial and temporal image transformations, simulating the variability expected during real-world ultrasound scanning. The 3D augmentation method enables SSL training on videos. The SSL algorithm is used to train a deep neural network model using unlabeled data.

Unlike standard supervised training, which maximizes agreement between model predictions and labels, the SSL algorithm disclosed herein maximizes agreement between visual representations from input video data undergoing different 3D augmentations, as described below. During the training process, the SSL algorithm thus learns to focus on regions of potential clinical relevance within the video and highlight these, ignoring the background. The SSL may be trained entirely using unlabeled data or using a combination of labeled and unlabeled data.

The specific mechanism of learning from unlabeled data are described below.

During training, a sub-clip of the ultrasound loop is extracted and applied with two different 3D augmentations. The two augmented sub-clips are then sent through a backbone network (for example, a convolutional neural network or CNN) to acquire 2D visual representations, then visual representations are converted to 1 dimensional projected features. The SSL training aims to maximize the agreement between these two projected features.

During deployment of the model (e.g., an inference mode or clinical usage mode), an ultrasound video is directly sent to the backbone network and the heat map is generated to locate and visualize clinical features. Importantly, this entire process can be trained using unlabeled data (with or without a small fraction of labeled data to provide supervision). As such, the method greatly reduces the need for expensive manual annotations.

The AI software developed using the SSL method produces heat maps that highlight the high probability (high activation) regions of the image. These heat maps are the output that is displayed to the user.

The present disclosure aids substantially in automated identification of pathologies, by improving the ability of machine learning systems to be trained using unlabeled data. Implemented on a processor in communication with an ultrasound imaging system or a database of stored radiology videos, the SSL ultrasound video feature detection system disclosed herein provides practical assistance to clinicians-especially those with limited training in identifying the features of concern. This improved feature identification transforms an expensive, time-consuming, labor-intensive training process into one that can be performed with little or no human oversight, and without the normally routine need for a human to label training videos frame by frame. This unconventional approach improves the functioning of the ultrasound imaging system, by providing detailed information about the identified pathologies in real time or near-real time.

The SSL ultrasound video feature detection system may be implemented as a process at least partially viewable on a display, and operated by a control process executing on a processor that accepts user inputs from a keyboard, mouse, or touchscreen interface, and that is in communication with one or more ultrasound sensors. In that regard, the control process performs certain specific operations in response to different inputs or selections made at different times. Certain structures, functions, and operations of the processor, display, sensors, and user input systems are known in the art, while others are recited herein to enable novel features or aspects of the present disclosure with particularity.

The application of machine learning to radiology images and videos (including but not limited to ultrasound images and videos), including feature detection, classification, regression, segmentation, and/or object detection.

These descriptions are provided for exemplary purposes only, and should not be considered to limit the scope of the SSL ultrasound video feature detection system. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

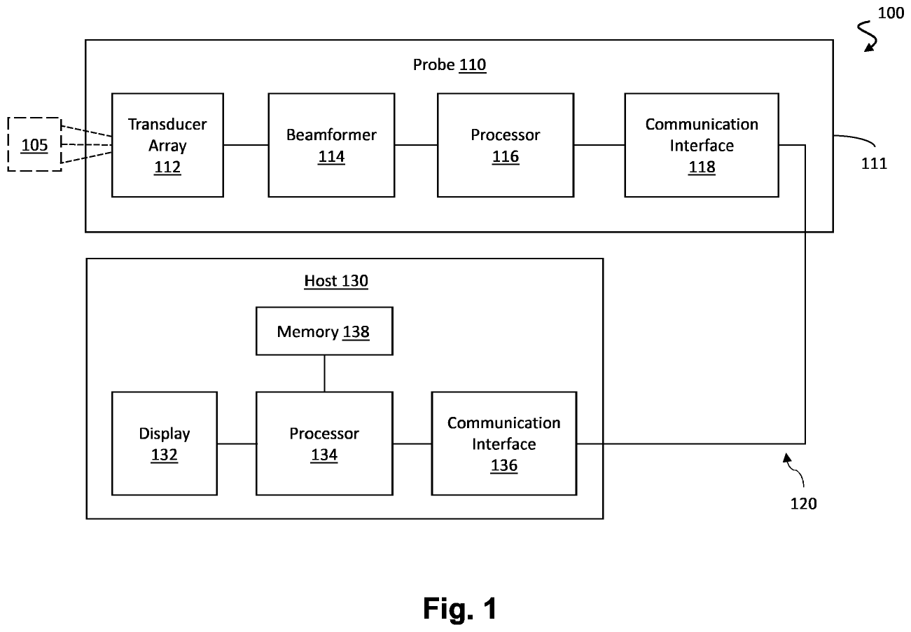

FIG. 1 is a schematic, diagrammatic representation of an ultrasound imaging system 100, in accordance with at least one aspect of the present disclosure. The ultrasound imaging system 100 may for example be used to acquire ultrasound video clips that may be used to train the SSL ultrasound video feature detection system, or that may be analyzed and highlighted in a clinical setting (whether in real time, near-real time, or as post-processing of stored video clips) by the SSL ultrasound video feature detection system.

The ultrasound imaging system 100 is used for scanning an area or volume of a subject's body. A subject may include a patient of an ultrasound imaging procedure, or any other person, or any suitable living or non-living organism or structure. The ultrasound imaging system 100 includes an ultrasound imaging probe 110 in communication with a host 130 over a communication interface or link 120. The probe 110 may include a transducer array 112, a beamformer 114, a processor circuit 116, and a communication interface 118.

The host 130 may include a display 132, a processor circuit 134, a communication interface 136, and a memory 138 storing subject information.

In some aspects, the probe 110 is an external ultrasound imaging device including a housing 111 configured for handheld operation by a user. The transducer array 112 can be configured to obtain ultrasound data while the user grasps the housing 111 of the probe 110 such that the transducer array 112 is positioned adjacent to or in contact with a subject's skin. The probe 110 is configured to obtain ultrasound data of anatomy within the subject's body while the probe 110 is positioned outside of the subject's body for general imaging, such as for abdomen imaging, liver imaging, etc. In some aspects, the probe 110 can be an external ultrasound probe, a transthoracic probe, and/or a curved array probe.

In other aspects, the probe 110 can be an internal ultrasound imaging device and may comprise a housing 111 configured to be positioned within a lumen of a subject's body for general imaging, such as for abdomen imaging, liver imaging, etc. In some aspects, the probe 110 may be a curved array probe. Probe 110 may be of any suitable form for any suitable ultrasound imaging application including both external and internal ultrasound imaging.

In some aspects, aspects of the present disclosure can be implemented with medical images of subjects obtained using any suitable medical imaging device and/or modality. Examples of medical images and medical imaging devices include x-ray images (angiographic images, fluoroscopic images, images with or without contrast) obtained by an x-ray imaging device, computed tomography (CT) images obtained by a CT imaging device, positron emission tomography-computed tomography (PET-CT) images obtained by a PET-CT imaging device, magnetic resonance images (MRI) obtained by an MRI device, single-photon emission computed tomography (SPECT) images obtained by a SPECT imaging device, optical coherence tomography (OCT) images obtained by an OCT imaging device, and intravascular photoacoustic (IVPA) images obtained by an IVPA imaging device. The medical imaging device can obtain the medical images while positioned outside the subject body, spaced from the subject body, adjacent to the subject body, in contact with the subject body, and/or inside the subject body.

For an ultrasound imaging device, the transducer array 112 emits ultrasound signals towards an anatomical object 105 of a subject and receives echo signals reflected from the object 105 back to the transducer array 112. The ultrasound transducer array 112 can include any suitable number of acoustic elements, including one or more acoustic elements and/or a plurality of acoustic elements. In some instances, the transducer array 112 includes a single acoustic element. In some instances, the transducer array 112 may include an array of acoustic elements with any number of acoustic elements in any suitable configuration. For example, the transducer array 112 can include between 1 acoustic element and 10000 acoustic elements, including values such as 2 acoustic elements, 4 acoustic elements, 36 acoustic elements, 64 acoustic elements, 128 acoustic elements, 500 acoustic elements, 812 acoustic elements, 1000 acoustic elements, 3000 acoustic elements, 8000 acoustic elements, and/or other values both larger and smaller. In some instances, the transducer array 112 may include an array of acoustic elements with any number of acoustic elements in any suitable configuration, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of acoustic elements (e.g., one or more rows, one or more columns, and/or one or more orientations) can be uniformly or independently controlled and activated. The transducer array 112 can be configured to obtain one-dimensional, two-dimensional, and/or three-dimensional images of a subject's anatomy. In some aspects, the transducer array 112 may include a piezoelectric micromachined ultrasound transducer (PMUT), capacitive micromachined ultrasonic transducer (CMUT), single crystal, lead zirconate titanate (PZT), PZT composite, other suitable transducer types, and/or combinations thereof.

The object 105 may include any anatomy or anatomical feature, such kidney, liver, and/or any other anatomy of a subject. The present disclosure can be implemented in the context of any number of anatomical locations and tissue types, including without limitation, organs including the liver, kidneys, gall bladder, pancreas, lungs; ducts; intestines; nervous system structures including the brain, dural sac, spinal cord and peripheral nerves; the urinary tract; as well as valves within the blood vessels, blood, abdominal organs, and/or other systems of the body. In some aspects, the object 105 may include malignancies such as tumors, cysts, lesions, hemorrhages, or blood pools within any part of human anatomy. The anatomy may be a blood vessel, as an artery or a vein of a subject's vascular system, including cardiac vasculature, peripheral vasculature, neural vasculature, renal vasculature, and/or any other suitable lumen inside the body. In addition to natural structures, the present disclosure can be implemented in the context of man-made structures such as, but without limitation, heart valves, stents, shunts, filters, implants and other devices.

The beamformer 114 is coupled to the transducer array 112. The beamformer 114 controls the transducer array 112, for example, for transmission of the ultrasound signals and reception of the ultrasound echo signals. In some aspects, the beamformer 114 may apply a time-delay to signals sent to individual acoustic transducers within an array in the transducer 112 such that an acoustic signal is steered in any suitable direction propagating away from the probe 110. The beamformer 114 may further provide image signals to the processor circuit 116 based on the response of the received ultrasound echo signals. The beamformer 114 may include multiple stages of beamforming. The beamforming can reduce the number of signal lines for coupling to the processor circuit 116. In some aspects, the transducer array 112 in combination with the beamformer 114 may be referred to as an ultrasound imaging component.

The processor 116 is coupled to the beamformer 114. The processor 116 may also be described as a processor circuit, which can include other components in communication with the processor 116, such as a memory, beamformer 114, communication interface 118, and/or other suitable components. The processor 116 may include a central processing unit (CPU), a graphical processing unit (GPU), a digital signal processor (DSP), an application specific integrated circuit (ASIC), a controller, a field programmable gate array (FPGA) device, another hardware device, a firmware device, or any combination thereof configured to perform the operations described herein. The processor 116 may also be implemented as a combination of computing devices, e.g., a combination of a DSP and a microprocessor, a plurality of microprocessors, one or more microprocessors in conjunction with a DSP core, or any other such configuration. The processor 116 is configured to process the beamformed image signals. For example, the processor 116 may perform filtering and/or quadrature demodulation to condition the image signals. The processor 116 and/or 134 can be configured to control the array 112 to obtain ultrasound data associated with the object 105.

The communication interface 118 is coupled to the processor 116. The communication interface 118 may include one or more transmitters, one or more receivers, one or more transceivers, and/or circuitry for transmitting and/or receiving communication signals. The communication interface 118 can include hardware components and/or software components implementing a particular communication protocol suitable for transporting signals over the communication link 120 to the host 130. The communication interface 118 can be referred to as a communication device or a communication interface module.

The communication link 120 may be any suitable communication link. For example, the communication link 120 may be a wired link, such as a universal serial bus (USB) link or an Ethernet link. Alternatively, the communication link 120 may be a wireless link, such as an ultra-wideband (UWB) link, an Institute of Electrical and Electronics Engineers (IEEE) 802.11 WiFi link, or a Bluetooth link.

At the host 130, the communication interface 136 may receive the image signals. The communication interface 136 may be substantially similar to the communication interface 118. The host 130 may be any suitable computing and display device, such as a workstation, a personal computer (PC), a laptop, a tablet, or a mobile phone.

The processor 134 is coupled to the communication interface 136. The processor 134 may also be described as a processor circuit, which can include other components in communication with the processor 134, such as the memory 138, the communication interface 136, and/or other suitable components. The processor 134 may be implemented as a combination of software components and hardware components. The processor 134 may include a central processing unit (CPU), a graphics processing unit (GPU), a digital signal processor (DSP), an application-specific integrated circuit (ASIC), a controller, an FPGA device, another hardware device, a firmware device, or any combination thereof configured to perform the operations described herein. The processor 134 may also be implemented as a combination of computing devices, e.g., a combination of a DSP and a microprocessor, a plurality of microprocessors, one or more microprocessors in conjunction with a DSP core, or any other such configuration. The processor 134 can be configured to generate image data from the image signals received from the probe 110. The processor 134 can apply advanced signal processing and/or image processing techniques to the image signals. In some aspects, the processor 134 can form a three-dimensional (3D) volume image from the image data. In some aspects, the processor 134 can perform real-time processing on the image data to provide a streaming video of ultrasound images of the object 105. In some aspects, the host 130 includes a beamformer. For example, the processor 134 can be part of and/or otherwise in communication with such a beamformer. The beamformer in the in the host 130 can be a system beamformer or a main beamformer (providing one or more subsequent stages of beamforming), while the beamformer 114 is a probe beamformer or micro-beamformer (providing one or more initial stages of beamforming).

The memory 138 is coupled to the processor 134. The memory 138 may be any suitable storage device, such as a cache memory (e.g., a cache memory of the processor 134), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, solid state drives, other forms of volatile and non-volatile memory, or a combination of different types of memory.

The memory 138 can be configured to store subject information, measurements, data, or files relating to a subject's medical history, history of procedures performed, anatomical or biological features, characteristics, or medical conditions associated with a subject, computer readable instructions, such as code, software, or other application, as well as any other suitable information or data. The memory 138 may be located within the host 130. Subject information may include measurements, data, files, other forms of medical history, such as but not limited to ultrasound images, ultrasound videos, and/or any imaging information relating to the subject's anatomy. The subject information may include parameters related to an imaging procedure such as an anatomical scan window, a probe orientation, and/or the subject position during an imaging procedure. The memory 138 can also be configured to store information related to the training and implementation of machine learning algorithms (e.g., neural networks) and/or information related to implementing image recognition algorithms for detecting/segmenting anatomy, image quantification algorithms, and/or image acquisition guidance algorithms, including those described herein.

The display 132 is coupled to the processor circuit 134. The display 132 may be a monitor or any suitable display. The display 132 is configured to display the ultrasound images, image videos, and/or any imaging information of the object 105.

The ultrasound imaging system 100 may be used to assist a sonographer in performing an ultrasound scan. The scan may be performed in a at a point-of-care setting. In some instances, the host 130 is a console or movable cart. In some instances, the host 130 may be a mobile device, such as a tablet, a mobile phone, or portable computer. During an imaging procedure, the ultrasound system can acquire an ultrasound image of a particular region of interest within a subject's anatomy. The ultrasound imaging system 100 may then analyze the ultrasound image to identify various parameters associated with the acquisition of the image such as the scan window, the probe orientation, the subject position, and/or other parameters. The ultrasound imaging system 100 may then store the image and these associated parameters in the memory 138. At a subsequent imaging procedure, the ultrasound imaging system 100 may retrieve the previously acquired ultrasound image and associated parameters for display to a user which may be used to guide the user of the ultrasound imaging system 100 to use the same or similar parameters in the subsequent imaging procedure, as will be described in more detail hereafter.

In some aspects, the processor 134 may utilize deep learning-based prediction networks to identify parameters of an ultrasound image, including an anatomical scan window, probe orientation, subject position, and/or other parameters. In some aspects, the processor 134 may receive metrics or perform various calculations relating to the region of interest imaged or the subject's physiological state during an imaging procedure. These metrics and/or calculations may also be displayed to the sonographer or other user via the display 132.

Before continuing, it should be noted that the examples described above are provided for purposes of illustration, and are not intended to be limiting. Other devices and/or device configurations may be utilized to carry out the operations described herein.

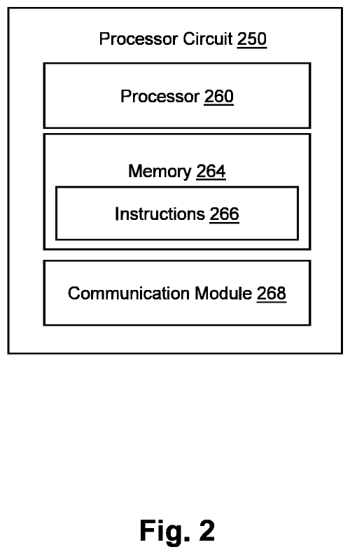

FIG. 2 is a schematic diagram of a processor circuit 250, according to aspects of the present disclosure. The processor circuit 250 may be implemented in the ultrasound imaging system 100, or other devices or workstations (e.g., third-party workstations, network routers, etc.), or on a cloud processor or other remote processing unit, as necessary to implement the method. As shown, the processor circuit 250 may include a processor 260, a memory 264, and a communication module 268. These elements may be in direct or indirect communication with each other, for example via one or more buses.

The processor 260 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 260 may also comprise another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 264 includes a non-transitory computer-readable medium. The memory 264 may store instructions 266. The instructions 266 may include instructions that, when executed by the processor 260, cause the processor 260 to perform the operations described herein. Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 250, and other processors or devices. In that regard, the communication module 268 can be an input/output (I/O) device. In some instances, the communication module 268 facilitates direct or indirect communication between various elements of the processor circuit 250 and/or the ultrasound imaging system 100. The communication module 268 may communicate within the processor circuit 250 through numerous methods or protocols. Serial communication protocols may include but are not limited to United States Serial Protocol Interface (US SPI), Inter-Integrated Circuit (I²C), Recommended Standard 232 (RS-232), RS-485, Controller Area Network (CAN), Ethernet, Aeronautical Radio, Incorporated 429 (ARINC 429), MODBUS, Military Standard 1553 (MIL-STD- 1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, model sharing between the processor and central server, or readings from the ultrasound imaging system 100) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

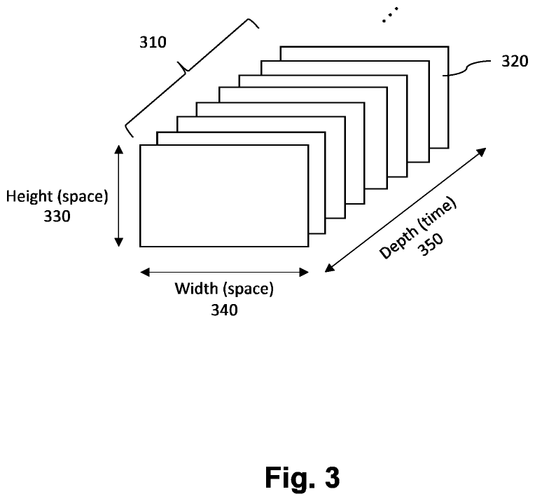

FIG. 3 is a schematic, diagrammatic representation of a radiology video, cineloop, or video clip 310 (e.g., an ultrasound video clip), in accordance with at least one aspect of the present disclosure. The ultrasound video clip 310 includes a number of frames 320. In an example, the ultrasound video clip 310 is between 1 second and 60 seconds long, at a frame rate of 30 frames per second, and may thus include between 30 and 1800 frames 320. Each frame as a Y-axis or height 330, and X-axis or width 340, which are spatial dimensions representing a 2D cross-section of the objects being imaged by the ultrasound imaging system. In addition, the ultrasound video clip 310 includes a depth or time axis 350, representing the times at which each frame 320 of the video clip 310 was captured. Thus, the ultrasound video clip 310 may be considered a 3D data structure. The video clip 310 can be any suitable modality with 2D image frames over time, such as x-ray, MRI, CT, etc.

In some aspects, the video clip 310 may include 4D data (X, Y, Z, time). For example, the 4D data can be 3D ultrasound (X, Y, Z are spatial dimensions)+time or other imaging modalities that are 3D (X, Y, Z are spatial dimension)+time, such as MRI, CT, etc. In other instances, the video clip 310 can include 4D multimodal/multi-imaging type images (X, Y are spatial dimensions in one imaging type of a modality+Z is imaging type dimension in the modality, with a different imaging type than X, Y dimensions+time). For example, the 4D multimodal/multi-imaging type images can be 2D ultrasound (X, Y are spatial dimensions in B-mode ultrasound)+Color Doppler ultrasound (Z)+time. In general, the “Z” dimension can be any suitable imaging type (e.g., Doppler, elastography, etc.) that is different than the X, Y dimensions (e.g., B-mode).

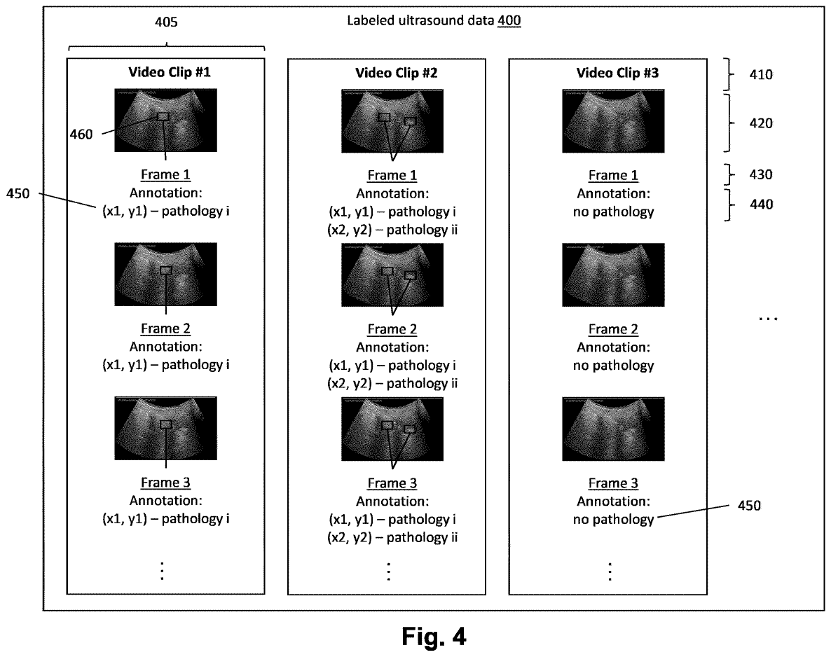

FIG. 4 is a schematic, diagrammatic representation of a labeled ultrasound data set 400, in accordance with at least one aspect of the present disclosure. The labeled ultrasound data set 400 includes a number of video clips 405. Each video clip 405 includes a title 410 and a plurality of frames 420. Each frame 420 includes a frame number 430 and an annotation 440. The annotation 440 may for example indicate whether or not there is a visible pathology in the frame 420. If a pathology is present, the annotation 440 may also include one or more pathology locations 450, and the frame 420 may include one or more bounding boxes 460 indicating those locations on the image.

Such frame-by-frame labeling is typically performed by hand, by a highly skilled clinician, in order to generate training data for traditional machine learning (ML) models.

However, given the large number of frames in even a short video clip, and given the high value of a trained clinician's time, such labeled ultrasound data sets 400 are expensive and labor-intensive to produce, and thus a limited amount of labeled data may be available for any given pathology, organ, or anatomical system.

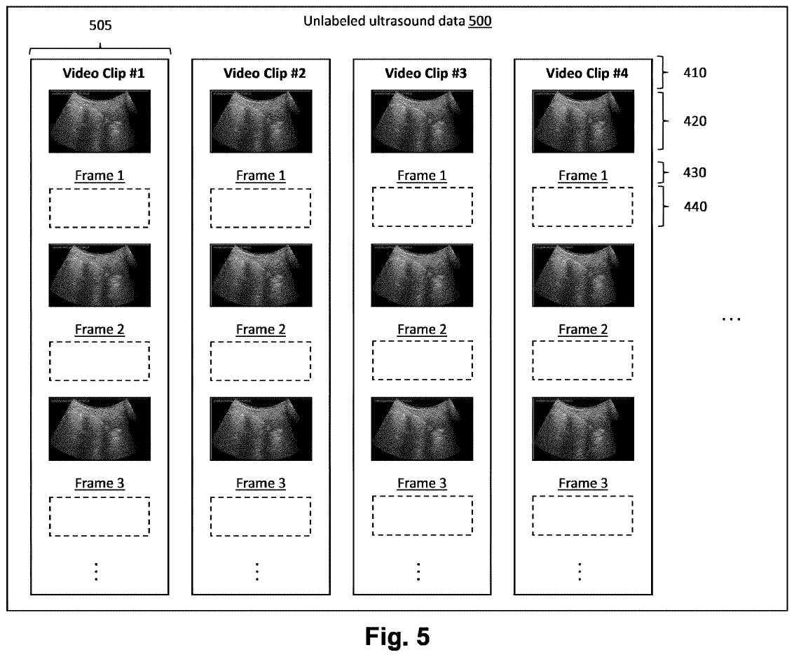

FIG. 5 is a schematic, diagrammatic representation of an unlabeled ultrasound data set 500, in accordance with at least one aspect of the present disclosure. The unlabeled ultrasound data set 500 includes a number of video clips 505. As with the labeled data set 400, each video 500 includes a title 410 and a plurality of frames 420, and each frame includes a frame number 430. However, the video clips 505 of the unlabeled data set 500 do not include an annotation 440 (or, alternatively, the annotation 440 is left blank). Unlabeled data 500 is easily acquired and stored, and may thus be cheaper and more readily available than labeled data 400. A given video clip 505 may include a pathology, or may include only healthy tissue. The video clips 505 should all be of the same anatomic system (e.g., all lung tissue), but can be captured at different positions, depths, angles, image settings, etc.

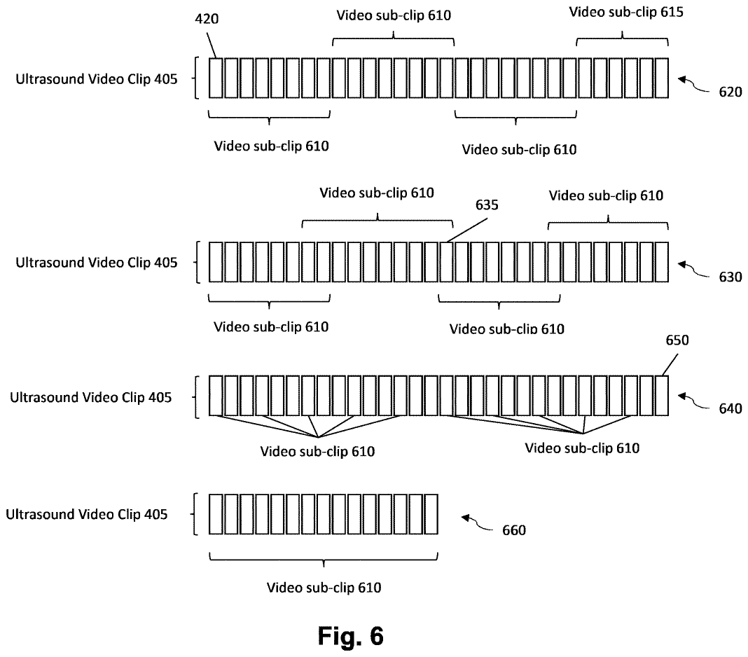

FIG. 6 is a schematic, diagrammatic representation of the extraction of sub-clips 610 from an ultrasound video clip 400, in accordance with at least one aspect of the present disclosure. In a first example 620, the video clip 405 comprises a plurality of frames 420, which are divided into sub-clips 610 of equal size, with a possible remainder clip 615 of smaller size. In example 620, the video clip 405 includes 30 frames, which are divided into three normal sub-clips 610 that each include eight frames 420. Eight frames may for example be a sub-clip size that is large enough to be useful for training an ML algorithm, but small enough to keep the computational burden within reasonable parameters. However, because 30 frames 420 are not evenly divisible by 8, the division leaves a remainder clip 615 of six frames.

In example 630, the video clip 405 includes 30 frames, which are divided into four sub-clips 610 that each include eight frames 420. To account for the fact that 30 is not evenly divisible by 8, each sub-clip 610 includes between 1 and 3 overlap frames 635, that are shared with another sub clip 610.

In example 640, in order to save data and speed up the training process, the 30-frame video clip 405 is divided into two five-frame sub-clips 610, each including only every third frame 420 from the video clip 405, such that two-thirds of the frames 420 are excluded frames 650.

In example 660, a 15-frame ultrasound video clip 405 is employed directly as a 15-frame sub-clip 610.

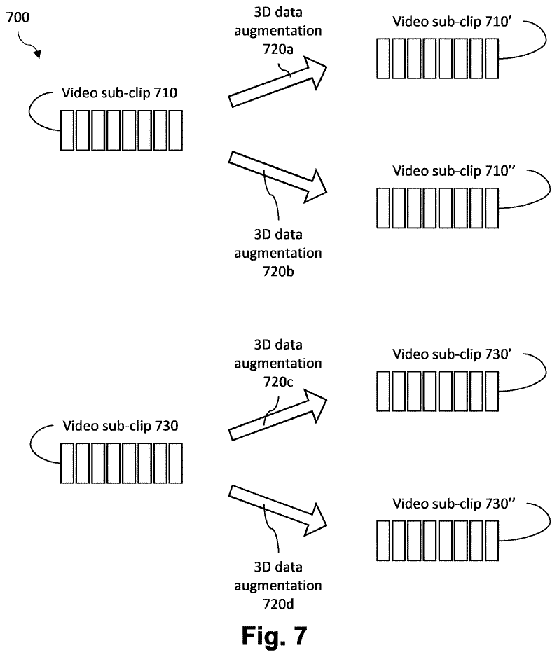

FIG. 7 is a schematic, diagrammatic illustration of a 3D spatial-temporal augmentation method 700 for ultrasound video data, in accordance with at least one aspect of the present disclosure. Augmentation is a key component of the SSL training procedures described below. For lung ultrasound and other radiology video data, the input is 3D data which requires a 3D data augmentation method.

In the example shown in FIG. 7, a sub-clip 710 is augmented using two different data augmentations 720a and 720b. The same first data augmentation 720a is applied to one, a plurality, or all of the frames in the sub-clip 710, thus generating a first augmented sub-clip 710′, and the second data augmentation 720b is applied to one, a plurality, or all of the frames in the sub-clip 710, thus generating a second augmented sub-clip 710″. In an example, the data augmentations are randomly selected from a list that may for example include: a random affine transform (e.g., a translation and/or rotation of the images), a random horizontal flip, a random color jitter (which may for example include random small color modifications to each pixel in the image, or changes to the entire image such as tint, gain, brightness, contrast, etc.), random noise (e.g., Gaussian or specular noise scattered throughout the image), random erasure of frames in the sub-clip, random time-reversal of frames in the sub-clip, random shuffling of frames in the sub-clip, or random dropping of some frames in the sub-clip and replacing them with the first frame. These augmentations are referred to as “3D” augmentations because the frames of the sub-clip are 2D images, and time serves as a third dimension.

In the example shown in FIG. 7, a second, different sub-clip 730 is augmented using two different, randomly selected data augmentations 720c and 720d, yielding two augmented video clips 730′ and 730″. Thus, it is understood that video clips 710, 710′, 710″, 730, 730′, and 730″ are all different from one another.

Applying these 3D augmentations to the sub-clip helps provide a large variety of extraneous data to the learning algorithm, such that the training process teaches the learning algorithm to distinguish clinically relevant details that might otherwise be lost amid noise, anatomical movement, movement of the ultrasound probe, and normal anatomy. Other numbers of data augmentations or other types of data augmentations may be used instead of or in addition to those described herein.

Affine transforms, flips, color jitter, and noise may be considered spatial augmentations, while frame deletions, time-reversal, and replacements may be considered temporal augmentations. One or multiple options are selected for each augmentation, so the combination of options leads to a diversity set of augmented video data to train the AI model using SSL. In other words, a randomly selected augmentation can include any combination of spatial and/or temporal augmentations.

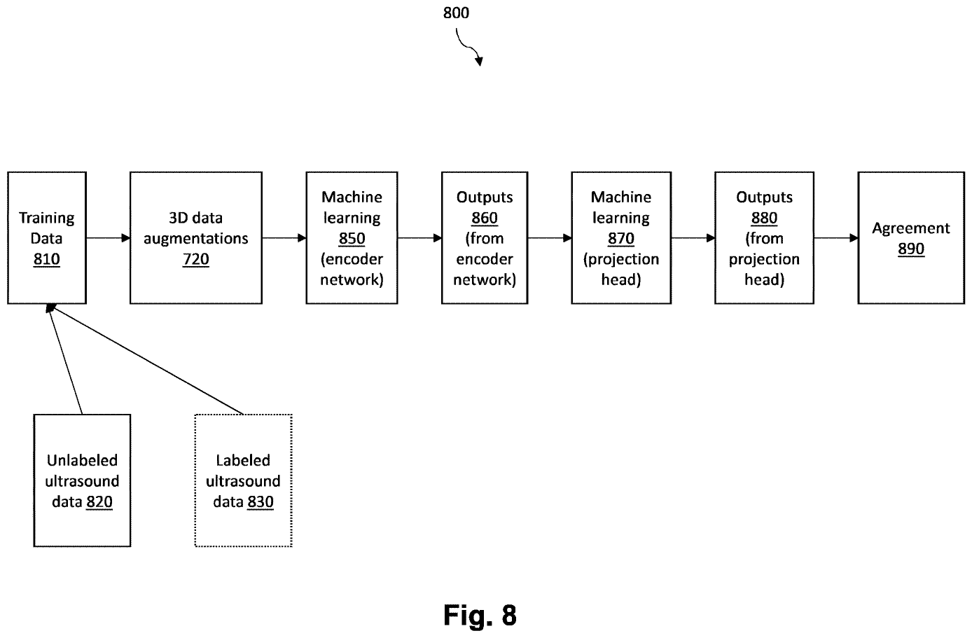

FIG. 8 is a schematic, diagrammatic overview, in block diagram form, of a self-supervised learning mode 800 for the SSL ultrasound video feature detection system, in accordance with at least one aspect of the present disclosure. In the example shown in FIG. 8, a set of training data 810 includes unlabeled ultrasound video data 820 and, optionally, a small amount of labeled ultrasound video data 830 (see FIG. 27, below). The amount of labeled data 830 can be relatively smaller than the amount of unlabeled data 820 (e.g., between 1% and 49%, between 1% and 25%, between 1% and 10%, of the total amount of data, and/or other values both larger and smaller). In some aspects, no labeled ultrasound data 830 is employed at all, such that all of the training data 810 is unlabeled data 820. The training data 810 is augmented with 3D data augmentations 720, and the resulting augmented data is fed into a machine learning model 850, such as a convolutional neural network (CNN) or encoder network.

Outputs 860 of the machine learning model 850 (e.g., a 2D matrix for each image frame indicating a level of clinical interest for different locations on the frame) are then fed into a second machine learning model 870 (or a different portion of the same learning model), which may for example be a multi-layer perceptron. The second machine-learning model 870 then turns the outputs 860 into outputs 880 (e.g., a single vector for each video clip, representing the heat map(s) of the video clip). These outputs 880 are then passed to an agreement step 890, which adjusts the weights of the machine learning model 850, 870 until a specified level of agreement is reached (as determined for example with a cosine similarity, mean square error, mutual information, absolute difference test, and/or other suitable metrics).

As will be appreciated by a person of ordinary skill in the art, the machine learning model/network 850 may implement or include any suitable type of learning network. For example, in some aspects, the machine learning model 850 could include a neural network, such as a convolutional neural network (CNN). In addition, the convolutional neural network may additionally or alternatively be or an encoder-decoder type network, or may utilize a backbone architecture based on other types of neural networks, such as an object detection network, classification network, etc. One example backbone network is the Darknet YOLO backbone, which can be used for object detection. The CNN may for example include a set of N convolutional layers, where N may be any positive integer. Fully connected layers can be omitted when the CNN is a backbone. The CNN may also include max pooling layers and/or activation layers. Each convolutional layer 520 may include a set of filters configured to extract features from an input (e.g., from a frame of the ultrasound video). The value N and the size of the filters may vary depending on the aspects. In some instances, the convolutional layers may utilize any non-linear activation function, such as for example a leaky rectified non-linear (ReLU) activation function and/or batch normalization. The max pooling layers gradually shrink the high-dimensional output to a dimension of the desired result (e.g., a heat map for model 850).

Fully connected layers may be referred to as perception or perceptive layers. In some aspects, perception/perceptive and/or fully connected layers may be found in the further machine learning model/network or projection head 870 (e.g., a multi-layer perceptron), to allow downstream processing (e.g., classification, regression, detection, segmentation, etc.). The network 870 may also include max pooling layers and/or activation layers. The max pooling layers gradually shrink the high-dimensional output to a dimension of the desired result (e.g., a heat map, a classification output, a regression output, a bounding box for a region of interest, etc., for the network 870).

These descriptions are included for exemplary purposes; a person of ordinary skill in the art will appreciate that other types of learning models, with features similar to or dissimilar to those described above, may be used instead or in addition, without departing from the spirit of the present disclosure. It is further noted that block diagrams are provided herein for exemplary purposes; a person of ordinary skill in the art will recognize myriad variations that nonetheless fall within the scope of the present disclosure. For example, block diagrams may show a particular arrangement of components, modules, services, steps, processes, or layers, resulting in a particular data flow. It is understood that some aspects of the systems disclosed herein may include additional components, that some components shown may be absent from some aspects, and that the arrangement of components may be different than shown, resulting in different data flows while still performing the methods described herein.

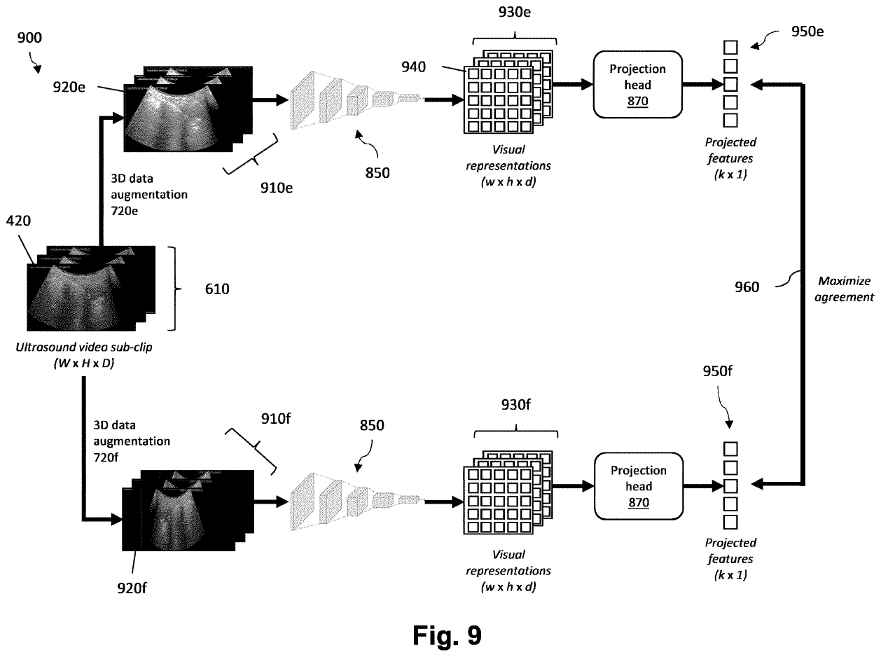

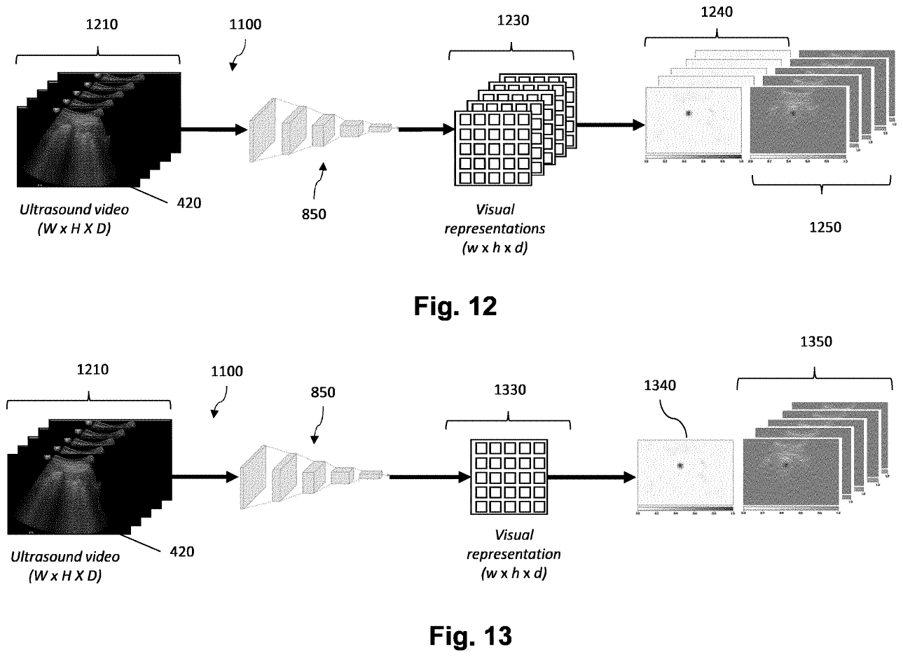

FIG. 9 is a is a schematic, diagrammatic overview, in block diagram form, of a self-supervised learning mode 900 for the SSL ultrasound video feature detection system, in accordance with at least one aspect of the present disclosure. The SSL ultrasound video feature detection system includes a training mode (shown here in FIG. 9) and an inference mode or clinical usage mode (shown below in FIGS. 12 and 13).

In an example, the training mode 900 may train a learning algorithm 850, which may for example be or include an artificial intelligence (AI), neural network, backbone network, or deep learning network such as a convolutional neural network (e.g., tiny Yolov3) algorithm. The training mode 900 may for example be executed on a computer with access to a database of stored video clips (e.g., ultrasound or other radiology clips). The training mode 900 may train the learning algorithm 850 using tens, hundreds, or thousands of video clips. However, the amount of data represented by these video clips may be substantial. For example, where the video clips are ultrasound videos of a patient's internal anatomy, each clip may include 0.2 seconds-60 seconds of video captured at 30 frames per second-- e.g., 30-1,800 separate images (image size is approximately several hundred pixels by several hundred pixels), each with a resolution of approximately 0.01 megapixels, 0.1 megapixels, 1 megapixels, 1.5 megapixels, and/or other value both larger and smaller. In an example, to limit the amount of time and computing power required to train the learning algorithm, the learning algorithm may be provided with a single sub-clip 610 at a time. In an example, the sub-clip 610 may include every third frame 420 of a 24-frame clip, for a total of 8 frames 420 of a single video) at a time.

Sub-clips may be selected randomly (which may be considered a form of data augmentation), or based on fixed parameters. During training, sub-clips are generated from full videos automatically as part of the data processing pipeline. In an example, the system may uniformly sample 8 frames from the video, based on a random first frame index i and fixed gap of frames X, selecting frame index i±n*X (n=0, 1, . . . ) so that in total 8 frames from the video are selected randomly by the computer. The video sub-clip has a width W (e.g., in pixels or millimeters), a height H (e.g., in pixels or millimeters), and a depth or duration D (e.g., in frames or milliseconds).

In the example shown in FIG. 9, the video sub-clip 610 is subjected to two different, randomly selected 3D data augmentations 720e and 720f, yielding two different augmented sub-clips 910e (comprising frames 920e) and 910f (comprising frames 920f).

Each augmented sub-clip 910e, 910f is then fed into the machine learning model 850, where each frame of the sub-clip is processed according to weights within the learning model 850, to yield respective visual representations 930e and 930f, each comprising a 2D matrix 940 for each frame of the respective video sub-clip. The 2D matrix 940 may for example be a w×h heat map identifying the locations within the image that contain suspected features of interest. In some cases, the 2D matrix may have the same resolution as the incoming frame or image (e.g., w=W and h=H). In other cases, the 2D matrix may have a smaller resolution in one or both of 2D dimensions (e.g., w<W and/or h<H). In an example, there may be down-sampled output for model 850, so that model 870 does not have too many parameters to train well. In some aspects, the depth d of the visual representations 931e, 930f (e.g., the number of matrices) is equal to the depth D of the ultrasound video sub-clip 610 (e.g., d=D). In other aspects, the depth d of the visual representations is less than the depth D of the ultrasound video sub-clip 610 (e.g., d<D), or is unity (e.g., d=1).

While FIG. 9 illustrates two instances of the model 850 and the model 870, it is understood that the self-supervised learning mode 900 includes one model 850 and one model 870 (as shown in, e.g., FIG. 8). Two instances of model 850 and model 870 are illustrated in FIG. 9 to more explicitly illustrate two branches in the training. As explained below, the training includes adjusting the values of the weights of the neural network based on agreement between the two branches (in contrast to agreement between labeled data and the output of the model 850 and/or model 870).

After all of the frames 920e, 920f of the sub-clip 910e, 910f have been fed through the machine learning model 850, there will be one matrix or visual representation 940 for each frame. These visual representations 930e, 930f are then processed by a projection head 870. The projection head 870 may for example be a fixed algorithm, or it may be a portion of the learning algorithm 850, such as a multi-layer perceptron. The projection head 870 reduces the visual representations (e.g., eight 2D matrices for each sub-clip) to a single “projected features” vector 950e, 950f representing the features identified in the augmented sub-clip. The vector 950e can be of size k×1. In one example, k=18. However, in other situations, k can be a human-assigned positive-integer parameter, and thus be values other than 18, fixed by the perceptron architecture. Depending on the implementation, too small a value for k is not representative for image features, while too large a value for k will increase model complexity/parameters.

The two projected features vectors 950e, 950f (e.g., the vector representing the first augmented sub-clip and the vector representing the second augmented sub-clip) are then compared in an agreement step 960, and the weights of the learning algorithm 850 are adjusted iteratively until the two projected features vectors 950e, 950f are within a specified level of agreement (e.g., as measured by a cosine similarity, mean square error, mutual information, absolute difference test, and/or other suitable metrics). The training is based on stochastic gradient descent, which means in each step sub-clips 610 are processed one by one, and weights are updated step by step from each sub-clip 610 to gradually improve agreement. Training of the learning algorithm may be considered complete when the level of agreement no longer improves by more than a threshold value as new sub-clips 610 are processed.

In some instances, the outputs of the model 850 (e.g., visual representations 930e, 930f) may not be suitable for direct comparison to one another in some instances. The machine learning model 870 generates outputs (two projected features vectors 950e, 950f) that can be compared to one another to determine the agreement. This is advantageous when training is completed using unlabeled data because the two projected features vectors 950e, 950f can be compared to one another (rather than comparing the output of the model 850 to labeled ultrasound data).

In an example, training of the machine learning model may be or include SimCLR (A Simple Framework for Contrastive Learning of Visual Representations) as the SSL training method, although other methods may be used instead or in addition. In cases where the video data is 4D rather than 3D, the spatial augmentation options need to be upgraded to be 3D rather than 2D spatial augmentations, but the overall flow remains the same.

In some instances, the machine learning model 850 and projection head 870 are combined here there are no intermediary outputs or visual representations 930e, 930f. Rather, the machine learning model 850 directly outputs the feature vectors 950e, 950f. This may be done, for example, when it is not desired for the system to generate heat maps.

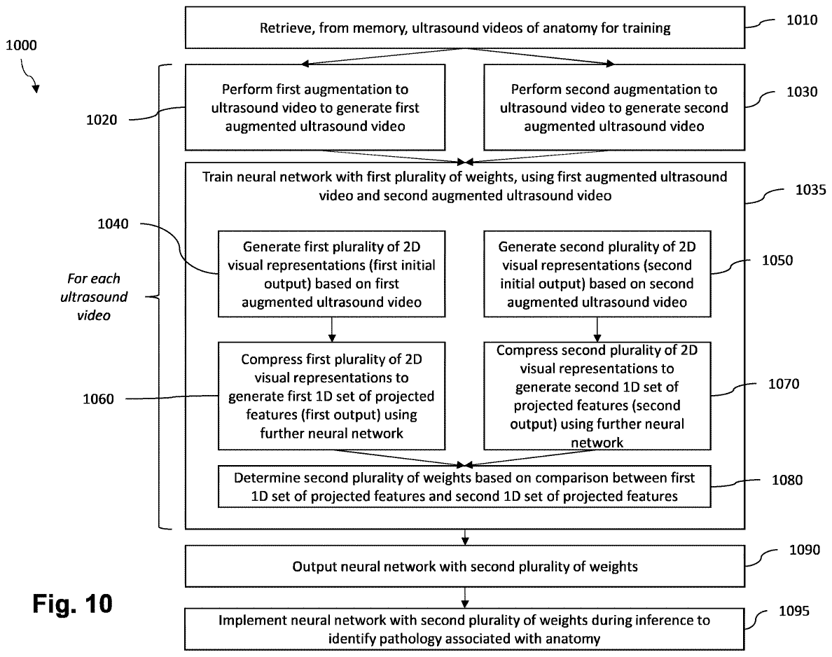

FIG. 10 is a flow diagram of an example SSL ultrasound video feature detection system training method 1000, according to at least one aspect of the present disclosure. It is understood that the steps of method 1000 may be performed in a different order than shown in FIG. 10, additional steps can be provided before, during, and after the steps, and/or some of the steps described can be replaced or eliminated in other aspects. One or more of steps of the method 1000 can be carried by one or more devices and/or systems described herein, such as components of the ultrasound imaging system 100 and/or processor circuit 250.

In step 1010, the method 1000 includes retrieving (e.g., from a memory or database) a number of ultrasound videos of the selected anatomy type, for training of the machine learning model. In some aspects, all of the ultrasound videos are unlabeled. In some aspects, a minority of the ultrasound videos are labeled. Each ultrasound video comprises a plurality of 2D frames, each frame representing a different time over a time period of the video. In some aspects, all frames of the plurality of frames are unlabeled. In some aspects, a minority of the frames of the plurality of frames are labeled. Examples of a video, video clip, or video sub-clip can be seen above in FIGS. 3-6.

Steps 1020 to 1070 may be performed repeatedly, on a large plurality of video clips or video sub-clips in order to fully train the machine learning algorithm or learning model.

In step 1020, the method 1000 includes performing a first, randomly selected augmentation to the currently selected ultrasound video, to generate a first augmented ultrasound video (see FIG. 7, above).

In step 1030, the method 1000 includes performing a second, randomly selected augmentation to the currently selected ultrasound video, to generate a second augmented ultrasound video (see FIG. 7, above).

In step 1035, the method 1000 includes training a neural network (e.g., model 850 in FIGS. 8 and 9) using the first and second augmented ultrasound video. The neural network initially includes a first plurality of weights (e.g., first plurality of values for the weights).

Step 1035 can include sub-steps 1040, 1050, 1060, 1070, and 1080.

In step 1040, the method 1000 includes generating a first plurality of 2D visual representations (e.g., a first initial output) based on the first augmented ultrasound video (see FIG. 9, above).

In step 1050, the method 1000 includes generating a second plurality of 2D visual representations (e.g., a second initial output) based on the second augmented ultrasound video (see FIG. 9, above).

In step 1060, the method 1000 includes compressing the first plurality of 2D visual representations to generate a first 1D set of projected features (e.g., a first feature vector or first output) using a further neural network (see model 870 in, e.g., FIGS. 8 and 9, above).

In step 1070, the method 1000 includes compressing the second plurality of 2D visual representations to generate a second 1D set of projected features (e.g., a second feature vector or second output) using the further neural network (see model 870 in, e.g., FIGS. 8 and 9, above).

In step 1080, the method 1000 includes determining a second plurality of weights (e.g., a second plurality of values for the weights) for the neural network and the further neural network, based on a comparison between the first 1D set of projected features and the second 1D set of projected features (see FIG. 9, above). The comparison can include a measure of agreement between the first 1D set of projected features and the second 1D set of projected features (see, e.g., agreement 890 in FIG. 8 and agreement 960 in FIG. 9).

Steps 1040 to 1080 may be performed iteratively on the first and second augmented ultrasound videos, continually updating and refining the values of the weights of the neural networks, until a desired level of agreement is reached between the first 1D set of projected features and the second 1D set of projected features (as measured for example with a cosine similarity, mean square error, mutual information, absolute difference test, and/or other suitable metrics).

In step 1090, the method 1000 includes providing or outputting the neural network with second plurality of weights. The second plurality of weights is an updated version (e.g., updated values) of the first plurality of weights, reflecting the iterative training of the network as additional video data is fed to it. During the iteration, the second values of the plurality of weights replaces the first values of the plurality of weights, and can then be replaced by a new “second” values of the plurality of weights in the next iteration of the loop. Providing or outputting the neural network with the second plurality of weights can include writing data to memory that is representative of the neural network with the second plurality of weights accessible in a memory (e.g., for implementation during inference, as in step 1095). Other examples of providing or outputting can include the processor circuit transmitting the data representative of the neural network with the second plurality of weights to a different computer or processor circuit. For example, training of the neural network can be performed by a manufacturer of an ultrasound system using a processor circuit at research & development or manufacturing location. The trained neural network (with the second plurality of weights) can be transmitted (online data transfer, data transfer via physical media, wired or wireless data transfer) to a different processor circuit that will be used to identify pathology in ultrasound video data of a patient. This different processor circuit can be an ultrasound console or another computer at a hospital, physician's office, or other clinical environment where patient data with potential pathology is being collected and/or evaluated.

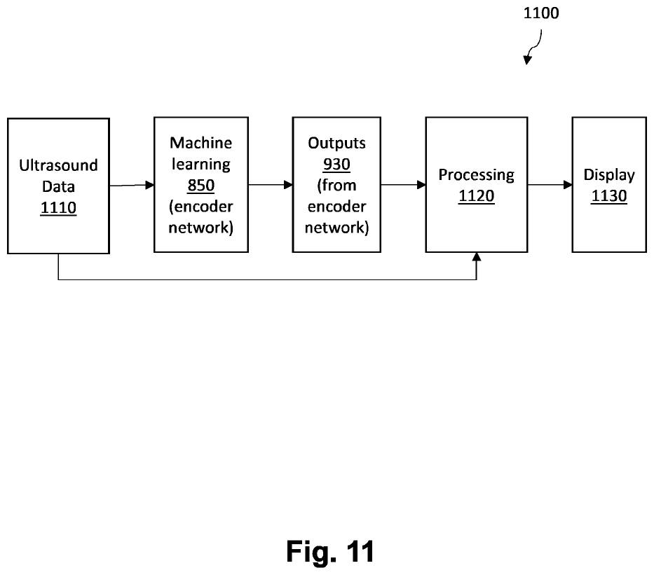

In step 1095, the method 1000 includes implementing the neural network, with the second plurality of weights, during inference (e.g., real-time annotation of ultrasound videos captured in a clinical setting) to identify pathologies associated with the studied anatomy (see FIGS. 11-15, below). In some instances, only the neural network (and not the further neural network) is used during inference. The neural network can be implemented, during inference, by the ultrasound console or another computer at a hospital, physician's office, or other clinical environment.

It is noted that flow diagrams are provided herein for exemplary purposes; a person of ordinary skill in the art will recognize myriad variations that nonetheless fall within the scope of the present disclosure. For example, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide each of the steps described herein into a plurality of machine instructions, and may execute these instructions at the rate of several hundred, several thousand, several million, or several billion per second, in a single processor or across a plurality of processors. Such rapid execution may be necessary in order to execute the method in real time or near-real time as described herein. For example, annotating ultrasound videos in real time may require analyzing a megapixel or more of data, thirty times per second.

FIG. 11 is a schematic, diagrammatic overview, in block diagram form, of an inference mode 1100 for the SSL ultrasound video feature detection system, in accordance with at least one aspect of the present disclosure. In the inference mode 1100 the trained machine learning algorithm or backbone network 850 is put to clinical use, such as embedded in one or more processors of an ultrasound imaging system 100. In this configuration, the learning algorithm or backbone network 850 is used to analyze clinical video streams such as ultrasound video, either in real time, near-real time, or post-processing of recorded video.

For example, a video (e.g., an ultrasound video, whether real-time or recorded) is directly sent to the trained learning algorithm or backbone network, which generates a visual representation or heat map for each frame of the video, to identify the locations of features that are suspected by the learning algorithm or backbone network to be of clinical interest.

In the example shown in FIG. 11, ultrasound video data 1110 (e.g., live video captured from a patient in real time, or recorded video captured from the patient at an earlier time) is fed into the trained machine learning algorithm 850, yielding outputs 930 (e.g., a heat map for each frame of the video 1110, often at a lesser resolution than the original video frames). Visual display of the heat maps is accomplished by a processing step 1120 (e.g., the encoder neural network and projection head 870 of FIG. 9) to up-sample the heat maps to the original resolution of the video frames. The heat maps indicate high-activation regions within the frames of the ultrasound video. For a well-performing SSL model, the highlighted regions of strong activation correlate with regions of clinical relevance (e.g. pathological areas within each frame of the video). The processing step 1120 also performs any other associated processing that gets the outputs 930 and or the ultrasound data 1110 ready for display such as transparency (e.g. the heat map could be transparent), changes in gain, brightness, or sharpness, smoothing over time (e.g., blending between frames), applying a color map or color key for display, etc. The processing step 1120 may also overlay the up-sampled heat maps directly onto the original video frames, thus yielding a highlighted video which is then displayed on a display 1130. In some aspects, the heat map and ultrasound video may be displayed side-by-side rather than overlaid, or in other configurations useful to the clinician.

FIG. 12 is a schematic, diagrammatic overview, in block diagram form, of an inference mode 1100 for the SSL ultrasound video feature detection system, in accordance with at least one aspect of the present disclosure. In the example shown in FIG. 12, an input video stream 1210 (whether real-time, near-real-time, or recorded), comprising a large plurality of frames 420, is fed into the machine learning algorithm 850, which produces visual representations (e.g., heat maps) 1230. In some cases, the heat maps may have the same resolution as the incoming video frames. In other cases, the heat maps may be up-sampled to match the resolution of the incoming video frames. In either case, the result is a flow of formatted heat maps 1240 that can be matched to the frames 420 of the incoming video stream 1210. The formatted heat maps 1240 can then be overlaid on the image frames 420 from which they were generated, yielding an output stream of highlighted video 1250. In an example, the learning algorithm may operate at a 30 Hz cycle, such that an incoming video stream captured at 30 Hz can be processed and highlighted in real time (e.g., with a delay of less than 33-milliseconds between the time the image frame is captured and the time it is highlighted with the heat map).

Importantly, this entire process can be trained using unlabeled data (with or without a small fraction of labeled data to provide supervision). As such, the method greatly reduces the need for expensive manual annotations.

FIG. 13 is a schematic, diagrammatic overview, in block diagram form, of an inference mode 1100 for the SSL ultrasound video feature detection system, in accordance with at least one aspect of the present disclosure. The data flow of FIG. 13 is similar to that of FIG. 12, except that only a single visual representation 1330 is generated, thus yielding a single formatted heat map 1340, which can be overlaid repeatedly on the frames 420 of the incoming video stream 1210, yielding the output stream of highlighted video 1350.

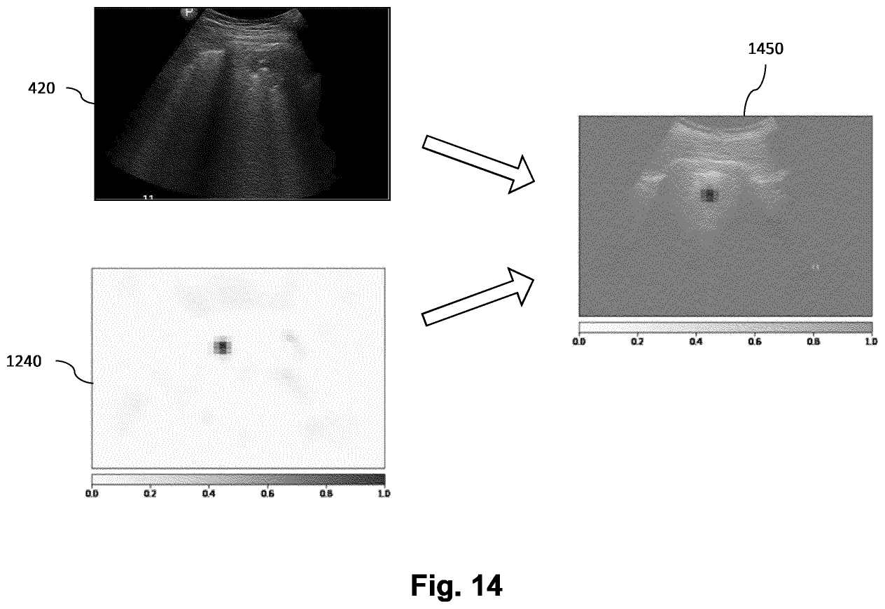

FIG. 14 is a schematic, diagrammatic view of a formatted heat map 1240 being overlaid on an incoming video frame 420 to yield a highlighted output frame 1450, in accordance with at least one aspect of the present disclosure.

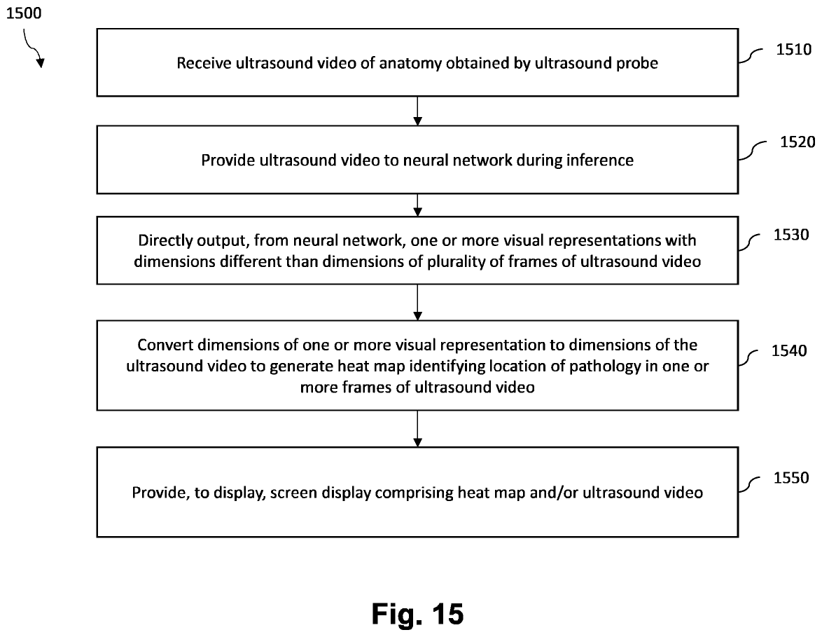

FIG. 15 is a flow diagram of an example inference-mode or clinical usage mode SSL video annotation method 1500, according to at least one aspect of the present disclosure. It is understood that the steps of method 1500 may be performed in a different order than shown in FIG. 15, additional steps can be provided before, during, and after the steps, and/or some of the steps described can be replaced or eliminated in other aspects. One or more of steps of the method 1500 can be carried by one or more devices and/or systems described herein, such as components of the ultrasound imaging system 100 and/or processor circuit 250.

In step 1510, the method 1500 includes receiving an ultrasound video of anatomy obtained by an ultrasound probe (e.g., in a clinical setting). The video may be real-time, near-real time, or may be stored in a volatile or non-volatile memory. In an example, the ultrasound video comprises a plurality of 2D frames over a time period of the ultrasound video. Examples of ultrasound videos, video clips, or video sub-clips can be found above, in FIGS. 3-6.

In step 1520, the method 1500 includes providing the ultrasound video to a neural network for inferencing, e.g., for highlighting of features in the ultrasound video to aid in clinical analysis (see FIG. 12, above).

In step 1530, the method 1500 includes directly outputting, from the neural network, one or more visual representations with dimensions identical to or different than the dimensions of plurality of frames of ultrasound video (see FIG. 12, above).

In step 1540, the method 1500 includes, if the dimensions of the visual representation are different than the dimensions of the plurality of frames, converting the dimensions of one or more visual representation to dimensions of the ultrasound video to generate a heat map (e.g., one heat map per frame, one heat map per several frames, one heat map for the entire video, etc.). The heat map identifies one or more locations of pathology in one or more frames of the ultrasound video (see FIG. 12, above). In some cases, the heat map identifies a location of a feature or anatomical feature that is suspected to have a pathology. In other cases, the heat map identifies no pathologies (e.g., if no pathologies are present in the image). In an example, the neural network trained with SSL does not know for sure what, if anything, is at the identified pixels (e.g., the dark spots of the heat maps).

Rather, the neural network trained with SSL knows that the image content of pixels (the dark spots of the heat maps) has something different compared to the image content in the other pixels. In some aspects, other neural networks (e.g., classification, trained with supervised learning using labeled data), can probabilistically identify if the dark spots in the heat maps have a pathology (or what kind of pathology). In some aspects, a further neural network configured for at least one of classification, regression, object detection, or segmentation.

These neural network are trained using supervised learning (i.e., labeled data), using the weights from the SSL training as a starting point.

In step 1550, the method 1100 includes providing, to a display, a screen display comprising the heat map and/or the ultrasound video. In some aspects, the heat maps are overlaid on the frames of the ultrasound video (see FIG. 14, above).

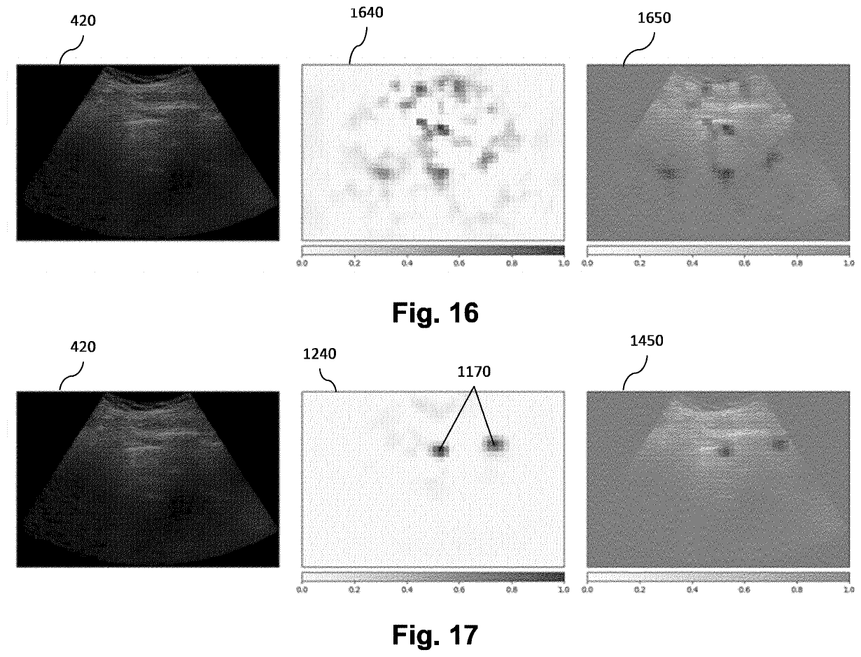

FIG. 16 is an exemplary representation of the highlighting of features in a video frame by a supervised learning algorithm, in accordance with at least one aspect of the present disclosure. In the example shown in FIG. 16, an input frame 420 is used to generate a formatted heat map 1640, which is then overlaid on the input frame 420 to yield an output frame 1650.

FIG. 17 is an exemplary representation of the highlighting of features in a video frame by a self-supervised learning (SSL) algorithm, in accordance with at least one aspect of the present disclosure. In the example shown in FIG. 16, an input frame 420 is used to generate a formatted heat map 1640, which is then overlaid on the input frame 420 to yield an output frame 1650.

In the particular example of FIGS. 16 and 17, the incoming video stream is ultrasound imagery of lung tissue containing consolidation (a pathological feature associated with pneumonia and other lung conditions). In FIG. 16, a standard deep learning AI model trained without SSL and using labeled data only, generates a heat map 1640 that is noisy and does not clearly identify the pathology, whereas in FIG. 17, the SSL ultrasound video feature detection system has clearly identified two regions 1710 that are likely consolidations. Thus, the SSL ultrasound video feature detection system shows better heat maps than the supervised learning process using human-labeled data.

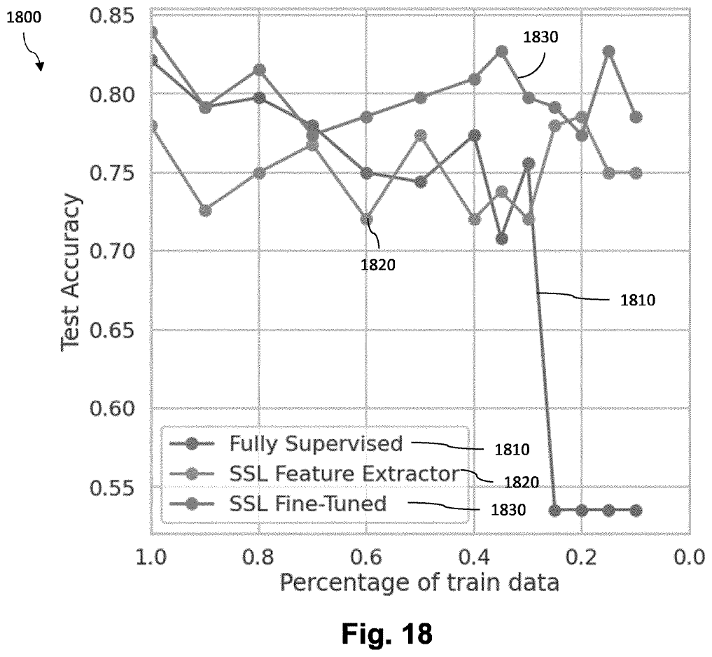

FIG. 18 is a graph 1800 showing test accuracy for partially trained machine learning models, in accordance with at least one aspect of the present disclosure. Three different machine learning models are represented: a fully supervised learning model 1810, an SSL feature extraction model 1820, and a fine-tuned SSL model 1830. Only labeled data is used for training the fully supervised learning model 1810. Only unlabeled data is used for training the SSL feature extraction model 1830. Both labeled data and unlabeled data are used for training the fine-tuned SSL model 1830.

The graph 1800 shows that, when fully trained with 100% of the necessary training data, the fine-tuned SSL model 1830 produces comparable results to the fully supervised learning model 1810, whereas the SSL feature extraction model 1820 is about 5% less accurate. However, when only 20% of the training data has been entered into the machine learning algorithm, the fine-tuned SSL model 1830 is already almost 80% accurate in identifying lung consolidations, whereas the fully supervised learning model 1810 has an accuracy of only 50%. Thus, the SSL ultrasound video feature detection system shows clear advantages over existing machine learning video annotation systems when large training datasets are not available.

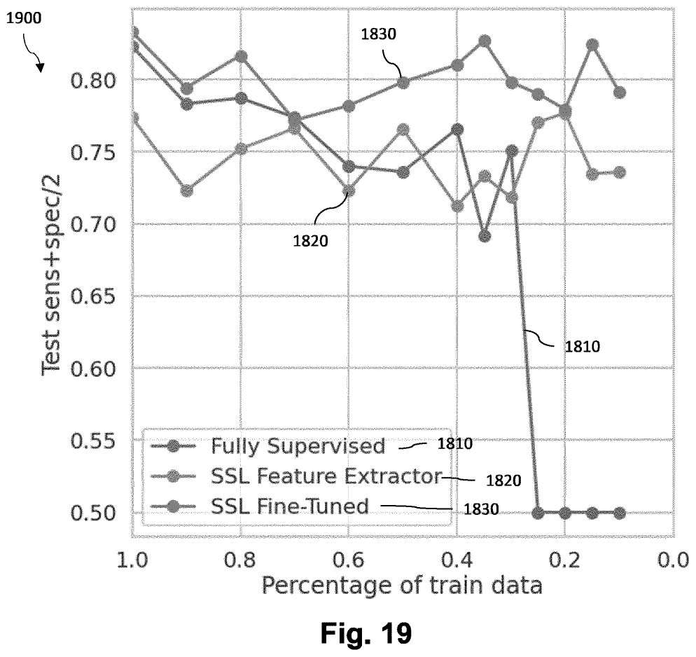

FIG. 19 is a graph 1900 showing the average of test sensitivity and test specificity for partially trained machine learning models, in accordance with at least one aspect of the present disclosure. As with FIG. 18, three different machine learning models are represented: a fully supervised learning model 1810, an SSL feature extraction model 1820, and a fine-tuned SSL model 1830. The graph 1800 shows that, when fully trained with 100% of the necessary training data, the fine-tuned SSL model 1830 produces comparable results to the fully supervised learning model 1810, whereas the SSL feature extraction model 1820 is about 5% less accurate. However, when only 20% of the training data has been entered into the machine learning algorithm, the fine-tuned SSL model 1830 is already almost 85% accurate in identifying lung consolidations, whereas the fully supervised learning model 1810 has an accuracy of only 50%. Thus, the SSL ultrasound video feature detection system shows clear advantages over existing machine learning video annotation systems when large training datasets are not available.

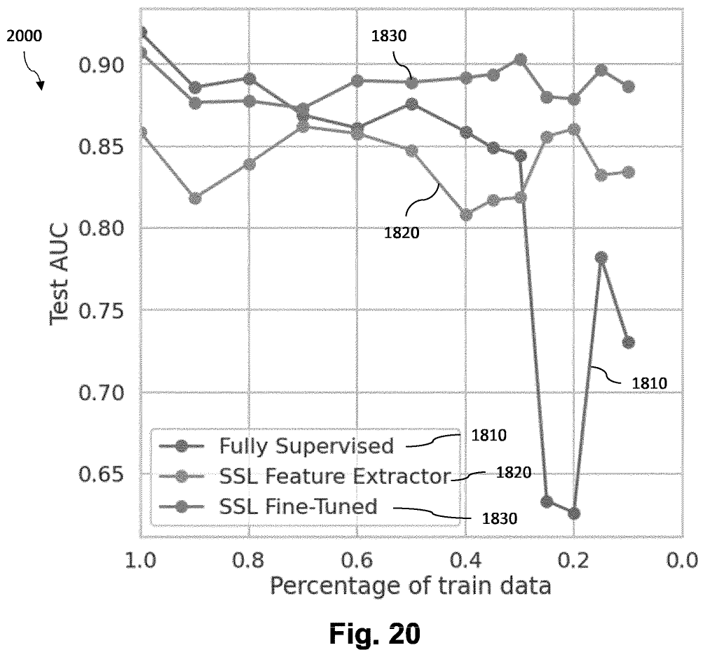

FIG. 20 is a graph 2000 showing test area under the curve (AUC) for partially trained machine learning models, in accordance with at least one aspect of the present disclosure. Three different machine learning models are represented: a fully supervised learning model 1810, an SSL feature extraction model 1820, and a fine-tuned SSL model 1830. The graph 1800 shows that, when fully trained with 100% of the necessary training data, the fine-tuned SSL model 1830 produces comparable results to the fully supervised learning model 1810, whereas the SSL feature extraction model 1820 is about 5% less accurate. However, when only 20% of the training data has been entered into the machine learning algorithm, the fine-tuned SSL model 1830 is already almost 95% accurate in identifying lung consolidations, whereas the fully supervised learning model 1810 has an accuracy of only about 60%. Thus, the SSL ultrasound video feature detection system shows clear advantages over existing machine learning video annotation systems when large training datasets are not available.

When the relative amount of labeled training data is high, the SSL methods have similar performance compared to a baseline (fully supervised) model. But when the amount of labeled training data is reduced (e.g. less than 30% of the total dataset), the benefits of using SSL approaches are more evident. The fully supervised method cannot be trained well with very limited data, so the test performance is poor. However, both SSL approaches can still work with minor changes in performance (i.e., accuracy remains for SSL fine-tuned method regardless of the amount of labeled data used).

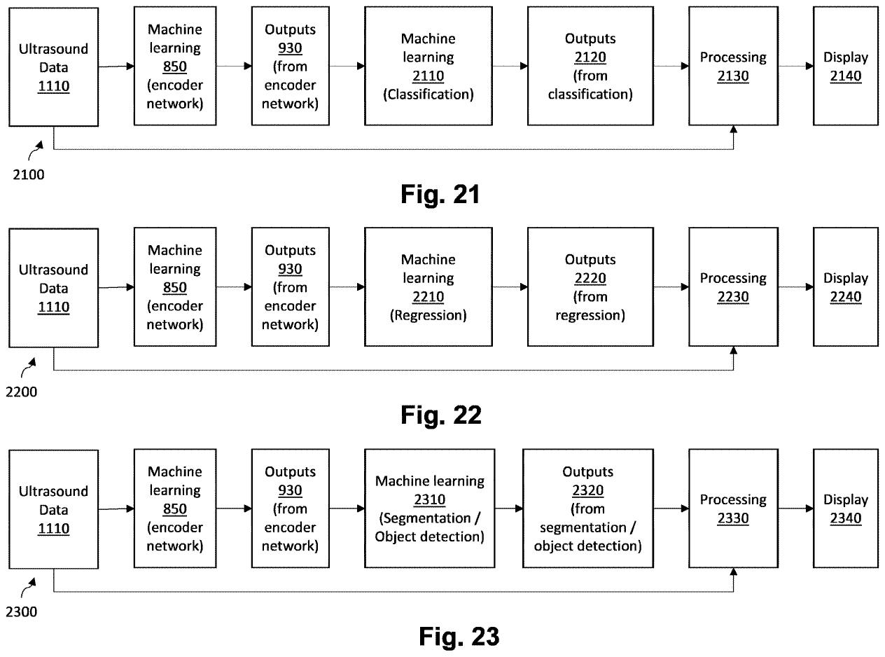

FIG. 21 is a schematic, diagrammatic overview, in block diagram form, of an inference mode 2100 for the SSL ultrasound video feature detection system, in accordance with at least one aspect of the present disclosure. The inference mode 2100 is similar to the inference mode 1100 shown in FIG. 11, except that the outputs 930 from the machine learning algorithm 850 are fed into a second machine learning algorithm 2110 (or a second portion of the machine learning algorithm 850) that serves as a classifier.

For example, a convolutional neural network (CNN) may additionally or alternatively be or include a multi-class classification network. The fully connected layers of the CNN may be non-linear and may gradually shrink the high-dimensional output to a dimension of the desired result (e.g., a classification output 2120). Thus, the fully connected layers may also be referred to as a classifier. A classification output may indicate a confidence score for each class based on the input image. A class indicating a high confidence score indicates that the input image or a section or pixel of the image is likely to include an anatomical object/feature of the class. Conversely, a class indicating a low confidence score indicates that the input image or a section or pixel of the image is unlikely to include an anatomical object/feature of the class.

The classification outputs 2120 are then included in the visual processing step 2130 that generates the highlighted video for the display 2140. An example output frame video including classifier outputs is shown below in FIG. 24. Examples of classification networks can be found for example in U.S. Provisional Patent Application No. 63/293,232, filed Dec. 23, 2021, entitled “Methods and systems for clinical scoring a lung ultrasound” and U.S. Provisional Patent Application No. 63/294501, filed Dec. 29, 2021, entitled “Machine-learning image processing independent of reconstruction filter”, each of which is incorporated by reference as though fully set forth herein.

In an example, a multi-class classification network may include an encoder path that processes an incoming image frame with convolutional layers such that the size is reduced. The resulting low dimensional representation of the image may be used to generate be used by the fully connected layers to regress and output one or more classes 542. In some regards, the fully connected layers may process the output of the encoder or convolutional layers. The fully connected layers 530 may additionally be referred to as task layers or regression layers, among other terms.

In some instances, the two machine learning algorithms 850 and 2110 may be combined, such that there is no intermediary output 930. Rather, the combined machine learning algorithm 850 may directly the processed images for display.

Referring again to FIG. 18, FIG. 19, and FIG. 20, the fully supervised learning model 1810, the SSL feature extraction model 1820, and the fine-tuned SSL model 1830 can be examples for providing the output of the SSL-trained neural network to a classifier neural network, as in FIG. 21. For example, during training, once the SSL is trained (e.g., neural network 850 and neural network 870 in FIG. 8 and FIG. 9), a fully connected layer after the output (e.g., of the neural network 870) to do classification. In this task, the fully supervised learning model 1810, the SSL feature extraction model 1820, and the fine-tuned SSL model 1830 can do the classification training depending on whether to use SSL trained weight as initial weights and whether to freeze the weights of neural network 850 and neural network 870. The fully supervised learning model 1810, the SSL feature extraction model 1820, and the fine-tuned SSL model 1830 used labeled classification labels for training. The fully supervised learning model 1810 did not use SSL trained weights and did not freeze the weights of neural network 850 and neural network 870 when training the classification neural network. The SSL feature extraction model 1820 did use SSL trained weights and did freeze the weights of neural network 850 and neural network 870 when training the classification neural network. The fine-tuned SSL model 1830 did use SSL trained weights and did not freeze the weights of neural network 850 and neural network 870 when training the classification neural network.

FIG. 22 is a schematic, diagrammatic overview, in block diagram form, of an inference mode 2200 for the SSL ultrasound video feature detection system, in accordance with at least one aspect of the present disclosure. The inference mode 2200 is similar to the inference mode 1100 shown in FIG. 11, except that the outputs 930 from the machine learning algorithm 850 are fed into a second machine learning algorithm 2210 (or a second portion of the machine learning algorithm 850) that serves as a regressor.

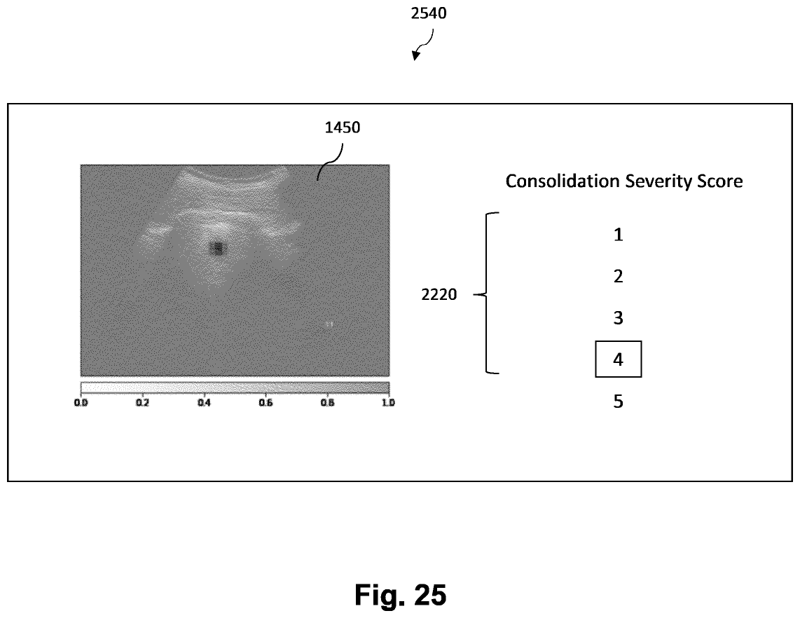

In an example, the fully connected layers of a CNN may be non-linear and may gradually shrink the high-dimensional output to a dimension of the desired result (e.g., a regression output 2220). The regression outputs 2220 are then included in the visual processing step 2230 that generates the highlighted video for the display 2240. An example output frame video including regression outputs is shown below in FIG. 25. An example of a regression network can be found in U.S. Provisional Patent Application No. 63/293,215, filed Dec. 23, 2021, entitled “Methods and systems for clinical scoring of a lung ultrasound”, which is incorporated by reference as though fully set forth herein.

In some instances, the two machine learning algorithms 850 and 2210 may be combined, such that there is no intermediary output 930. Rather, the combined machine learning algorithm 850 may directly the processed images for display.

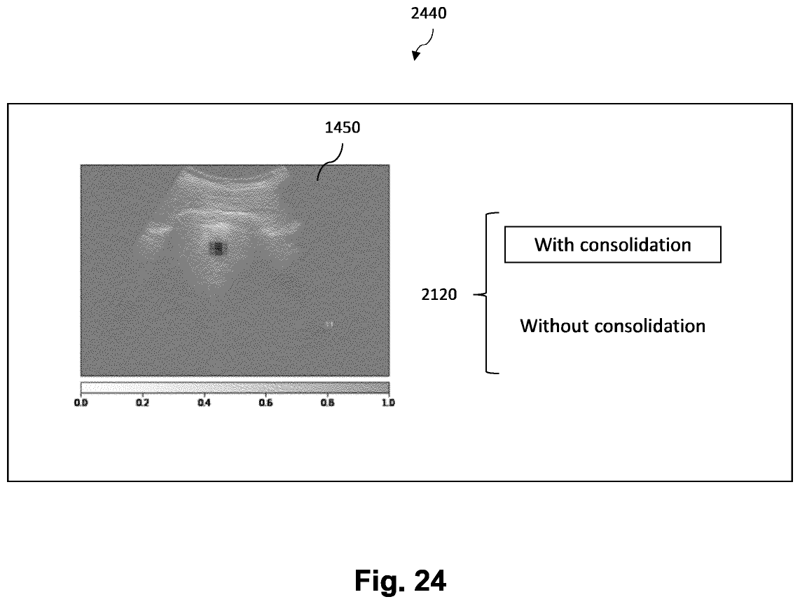

FIG. 23 is a schematic, diagrammatic overview, in block diagram form, of an inference mode 2300 for the SSL ultrasound video feature detection system, in accordance with at least one aspect of the present disclosure. The inference mode 2300 is similar to the inference mode 1100 shown in FIG. 11, except that the outputs 930 from the machine learning algorithm 850 are fed into a second machine learning algorithm 2310 (or a second portion of the machine learning algorithm 850) that serves as an object detector, bounding box generator, or segmentor/segmenter.

A heat map may for example show the relative importance of different regions of the image (typically at a pixel-by-pixel level) as a function of the model input. Heat maps are not necessarily specific to a target class or classes, although they could be if the mechanism for generating the heat map is conditioned on classes. Conversely, a bounding box indicates the “positive” regions in which objects of a target class or classes are detected. Bounding boxes do not provide pixel-by-pixel information across the entire image. An example of bounding box object detection can be found in India Patent Application No. 202141034243, filed Jul. 29, 2021, entitled “Generating location data” (International Application No. PCT/EP2022/070410), which are incorporated by reference as though fully set forth herein.

A related output type is segmentation, which is similar to a binarized version of the heat map or a pixel-by-pixel implementation of the bounding box. Segmentations are also pixel-by-pixel, with each pixel classified into one or more of the target classes. Thus, the output is a pixel mask that “color codes” different regions based on which class they belong to. Examples of image or video segmentation can be found for example in U.S. Provisional Patent Application No. 63/325,660, filed Mar. 31, 2022, entitled “Methods and systems for ultrasound-based structure localization using image and user inputs”, and U.S. Publication No. 2022/0198669, entitled “Segmentation and view guidance in ultrasound imaging and associated devices, systems, and methods”, each of which is incorporated by reference as though fully set forth herein.

In addition, the convolutional neural network may additionally or alternatively be trained to identify features within an image. In an example, the weights for SSL trained encoder neural network can also be a good initialization of downstream feature video detection tasks (e.g. bounding box detection), which is another important task for ultrasound applications. The fully connected layers of the CNN may be non-linear and may gradually shrink the high-dimensional output to a dimension of the desired result (e.g., a bounding box output 2320).

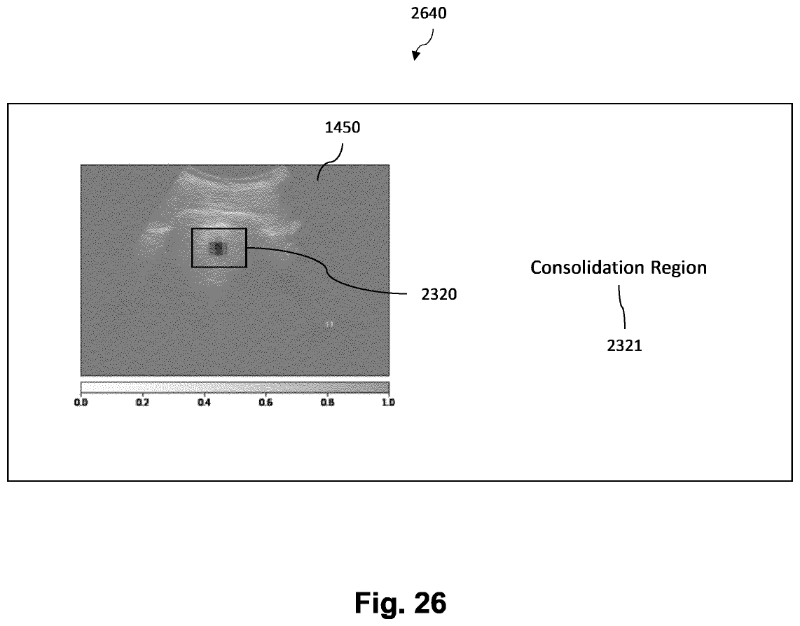

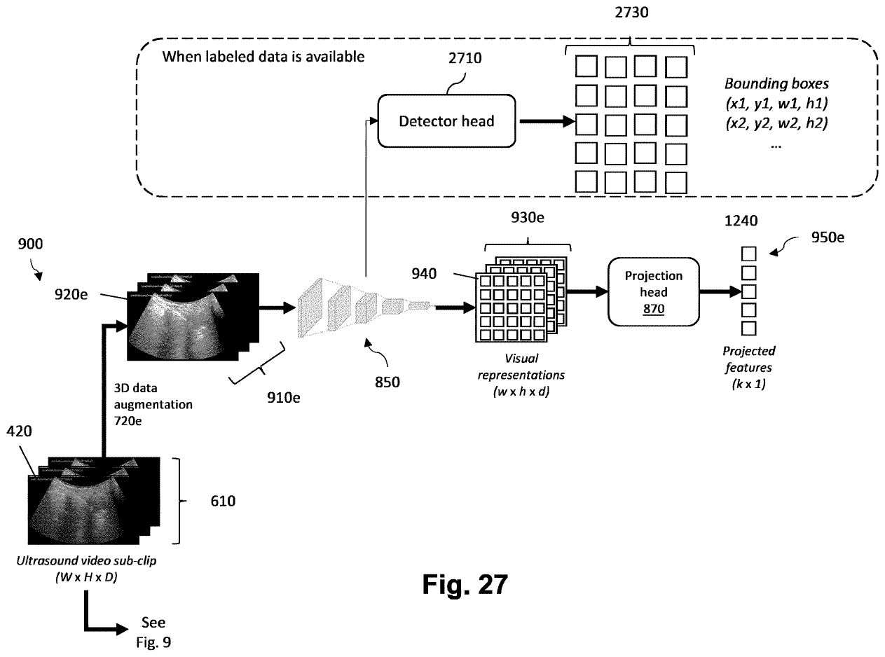

The bounding box 2320 are then included in the visual processing step 2330 that generates the highlighted video for the display 2340. An example output frame video including bounding box outputs is shown below in FIG. 26.

Thus, the encoder neural network may be followed by object detection layers (e.g. based on YOLO) can be trained to detect consolidation regions given bounding box labels.

The SSL trained encoder contains meaningful visual representation, which helps the network to locate important regions from the ultrasound video, and thus outperforms the baseline detection model if trained from scratch on the detection performance.

In some instances, the two machine learning algorithms 850 and 2310 may be combined, such that there is no intermediary output 930. Rather, the combined machine learning algorithm 850 may directly the processed images for display.

FIG. 24 is an example screen display 2440 that includes both the highlighted image frame 1450 and a classification output 2120, in accordance with at least one aspect of the present disclosure. The classification output may for example report whether or not a given anatomy or pathology is believed to be present in the image frame.

FIG. 25 is an example screen display 2540 that includes both the highlighted image frame 1450 and a regression output 2220 (e.g., a numerical score indicating the probability or severity of a detected pathology), in accordance with at least one aspect of the present disclosure. The regression output may for example report a severity value (on in a range of values) of a given pathology believed to be present in the image frame.

FIG. 26 is an example screen display 2640 that includes both the highlighted image frame 1450 and an object detection output, in accordance with at least one aspect of the present disclosure. The object detection output may for example be or include a bounding box 2320 indicating a region of interest and/or a label 2321 identifying the region of interest.

FIG. 27 is a schematic, diagrammatic overview, in block diagram form, of at least a portion of a training mode 900 for the SSL ultrasound video feature detection system, in accordance with at least one aspect of the present disclosure. The data flow of FIG. 27 is similar to that of FIG. 9, except that for clarity, only one branch (e.g., 3D augmentation 720e) is shown.

However, the training mode 900 of FIG. 27 includes training when some labeled data is included. The labeled data can include annotations with labeled boxes in one or more frames of the input video 420 (see, e.g., FIG. 4). In such cases, the machine learning algorithm 850 passes the labeled data to a detector head 2710 (e.g., a YoloV3 learning network, a neural network, or other machine learning algorithm), which outputs a series of bounding boxes 2730, each having an x position, y position, width, and height (e.g., all measured in pixels or millimeters). Each bounding box 2730 can also include a confidence level or confidence score. These bounding boxes are then used for joint agreement and detection loss in training, though not in inference mode.

For the detector/labeled data branch, the output is the bounding box predictions (x, y, width, height). These predicted boxes are compared with ground truth boxes using IOU loss (metric to measure overlaps of boxes). During this training, the weights of CNN (850) need to be adjusted to maximize the overlap, meanwhile maximizing the agreement between outputs from two unlabeled branches.

When both labeled data and unlabeled data are available, the training mode 900 has two parts of loss to optimize (loss of detector head 2710 from labeled boxes of labeled data and SSL loss on consistency from two branches of unlabeled data). Two losses are added together for training the model. Training minimizes the summed loss by adjusting the weight values of the neural network(s). In some instances, weights (e.g., to multiplied to a given loss, different than the weight values of the neural networks) may be added to control the portion of contribution from each part of the loss, so that the sum of the losses becomes weighted sum.

Although not shown in FIG. 27, these features (when present) are replicated for the other branch of FIG. 9 (e.g., 3D augmentation 720f).

As will be readily appreciated by those having ordinary skill in the art after becoming familiar with the teachings herein, the SSL ultrasound video feature detection system advantageously permits a highly accurate video annotation machine learning algorithm to be trained using predominantly or exclusively unlabeled training data, in shorter time and with less labor and expense.

A number of variations are possible on the examples and aspects described above. For example, the systems, methods, and devices described herein are not limited to lung ultrasound applications. Rather, the same technology can be applied to images of other organs or anatomical systems such as the heart, brain, digestive system, vascular system, etc. Furthermore, the technology disclosed herein is also applicable to other medical imaging modalities where 3D data is available, such as other ultrasound applications, camera-based videos, X-ray videos, and 3D volume images, such as computer aided tomography (CT) scans, magnetic resonance imaging (MRI) scans, optical coherence tomography (OCT) scans, or intravenous ultrasound (IVUS) pullback sequences. The output of the SSL-trained AI model are heat maps highlighting regions of strong network activation associated with features of clinical relevance (e.g. pathology) in the cineloop. The heat map outputs are readily detectable. 3D augmented cineloops can be provided to the SSL-trained AI to test whether the model predictions are sensitive to these augmentations. If the AI predictions are robust against these 3D augmentations, there is a strong likelihood that 3D spatial and temporal augmentations were used. The technology described herein can be used in a variety of settings including emergency department, intensive care, inpatient, and out-of-hospital settings.

Accordingly, the logical operations making up the aspects of the technology described herein are referred to variously as operations, steps, objects, layers, elements, components, algorithms, or modules. Furthermore, it should be understood that these may occur or be performed or arranged in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the SSL ultrasound video feature detection system. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of the SSL ultrasound video feature detection system as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. A system, comprising:
a display; and
a processor configured for communication with the display, wherein the processor is configured to:
receive an ultrasound video of anatomy obtained by an ultrasound probe, wherein ultrasound video comprises a plurality of frames;
provide the ultrasound video to a neural network during inference, wherein the neural network is trained with self-supervised learning using unlabeled ultrasound videos comprising at least one of a plurality of spatial augmentations or a plurality of temporal augmentations;
generate, using the neural network, a heat map identifying a location of a pathology in one or more frames of the plurality of frames; and
provide, to the display, a screen display comprising the heat map.

2. The system of claim 1, wherein the neural network is trained using only unlabeled ultrasound videos.

3. The system of claim 1, wherein the screen display further comprises the ultrasound video.

4. The system of claim 3, wherein the heat map is overlaid on the one or more frames of the plurality of frames of the ultrasound video.

5. The system of claim 4,
wherein the heat map comprises a single heat map,
wherein the single heat map is overlaid on each of the plurality of frames of the ultrasound video.

6. The system of claim 4,
wherein the processor is configured to generate a plurality of heat maps, and
wherein the screen display comprises the plurality of heat maps respectively overlaid on plurality of frames of the ultrasound video.

7. The system of claim 1,
wherein a direct output of the neural network comprises a visual representation associated the one or more frames of the ultrasound video, and
wherein dimensions of the visual representation are different than dimensions of the plurality of frames of the ultrasound video.

8. The system of claim 7, wherein, to generate the heat map, the processor is configured to convert the dimensions of the visual representation to the dimensions of the ultrasound video.

9. The system of claim 7, wherein the processor is configured to:
provide the visual representation to a further neural network configured for at least one of classification, regression, object detection, or segmentation; and
generate an output using the further neural network, wherein the output is associated with at least one of the classification, the regression, the object detection, or the segmentation, and wherein the output is different than the visual representation and the heat map; and
wherein the screen display comprises a visualization based on the output.

10. The system of claim 1,
further comprising the ultrasound probe,
wherein the processor is configured to control the ultrasound probe to obtain the ultrasound video.

11. A system, comprising:
a memory comprising an ultrasound video of anatomy obtained by an ultrasound probe, wherein the ultrasound video is unlabeled and comprises a plurality of frames;
a processor configured for communication with the memory, wherein the processor is configured to:
retrieve the ultrasound video from the memory;
perform a first augmentation to the ultrasound video to generate a first augmented ultrasound video;
perform a second augmentation to the ultrasound video to generate a second augmented ultrasound video, wherein the first augmentation and the second augmentation comprise at least one of a spatial augmentation to the plurality of frames or a temporal augmentation to the plurality of frames;
train a neural network with a first plurality of weights, using the first augmented ultrasound video and the second augmented ultrasound video, wherein the training of the neural network comprises self-supervised learning; and
provide, after the training, the neural network with a second plurality of weights, wherein the neural network with the second plurality of weights is configured to be implemented during inference to identify pathology associated with the anatomy,
wherein, to train the neural network, the processor is configured to:
generate a first output associated with the neural network based on the first augmented ultrasound video;
generate a second output associated with the neural network based on the second augmented ultrasound video; and
determine the second plurality of weights based on a comparison between the first output and the second output.

12. The system of claim 11, wherein, to train the neural network, the processor is configured to implement a first processing path associated with first augmented ultrasound video and a second processing path associated with the second augmented ultrasound video.

13. The system of claim 11,
wherein the memory further comprises a plurality of ultrasound videos,
wherein the processor is configured to:
obtain the plurality of ultrasound videos from the memory; and
train the neural network based on the plurality of ultrasound videos, and wherein a majority of the plurality of ultrasound videos are unlabeled.

14. The system of claim 13, wherein all of the plurality of ultrasound videos are unlabeled.

15. The system of claim 11,
wherein the spatial augmentation comprises a change to how image content is depicted in the plurality of frames, and
wherein the temporal augmentation comprises a change to an order in which the plurality of frames are arranged.

16. The system of claim 11,
wherein the first augmentation is different than the second augmentation, and
wherein the ultrasound video, the first augmented ultrasound video, and the second augmented ultrasound video are different from one another.

17. The system of claim 11,
wherein the first output associated with the neural network comprises a first one-dimensional (1D) set of projected features,
wherein the second output associated with the neural network comprises a second 1D set of projected features, and
wherein the processor is configured to determine the second plurality of weights to maximize agreement between the first 1D set of projected features and the second 1D set of projected features.

18. The system of claim 17,
wherein, to train the neural network, the processor is configured to:
generate a first initial output of the neural network based on the first augmented ultrasound video, before the first output is generated; and
generate a second initial output of the neural network based on the second augmented ultrasound video, before the second output is generated,
wherein the first initial output comprises a first plurality of two-dimensional (2D) visual representations, and
wherein the second initial output comprises a second plurality of 2D visual representations.

19. The system of claim 18, wherein, to train the neural network, the processor is configured to:
compress the first plurality of 2D visual representations using a further neural network to generate the first 1D set of projected features, and
compress the second plurality of 2D visual representations using the further neural network to generate the second 1D set of projected features.

20. The system of claim 19,
wherein the neural network comprises a convolutional neural network, and
wherein the further neural network comprises a multilayer perceptron.
